(19) United States
(12) Patent Application Publication
(10) Pub. No.: US 2008/0247991 A1
(43) Pub. Date: Oct. 9, 2008

(54) Solution Additives For the Attenuation of Protein Aggregation

(76) Inventors: Bernhardt L. Trout, Cambridge, MA (US); Daniel I.C. Wang, Newton, MA (US); Brian M. Baynes, Somerville, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/590,827

(22) PCT Filed: Feb. 28, 2005
(86) PCT No.: PCT/US05/06603

§ 371 Date: Aug. 16, 2007

Related U.S. Application Data
(60) Provisional application No. 60/547,969, filed on Feb. 26, 2004.

(51) Int. Cl. A61K 38/21; C07H 3/02; C07C 31/18; C13K 13/00; C13K 1/00; C07C 279/14; C07D 207/16; C07D 239/28; C07C 233/47; G06F 19/00; C08G 65/02; C08G 65/34; C08G 69/26; C07K 14/505; C07K 14/555; C07K 1/107; C07K 16/00; C07K 14/62; A61K 38/02; A61K 39/395; A61K 38/28; A61K 38/22

(52) U.S. Cl. 424/85.4; 536/1.11; 568/852; 536/123.13; 564/240; 548/535; 544/335; 564/153; 702/22; 702/19; 530/410; 530/390.5; 530/303; 514/2; 424/130.1; 424/133.1; 514/8; 530/351; 530/397; 528/332; 528/425; 528/417; 526/319

(57) ABSTRACT

In part, the present invention relates to a compound or polymer comprising a non-protein-binding moiety and at least one protein-binding group. The present invention relates to a method of screening compounds or polymers for the property of inhibiting protein aggregation in solution, a method of preparing a compound or polymer having the property of protein aggregation inhibition in solution, a method of classifying a compound or polymer as either inhibitory of protein aggregation in solution or not inhibitory of protein aggregation in solution, and to a method of determining the preferential binding coefficient, Γ_{XP}, of an additive in a protein solution. The present invention also relates to a method of suppressing or preventing aggregation of a protein in solution, a method of decreasing the toxicological risk associated with administering a protein to a mammal in need thereof, and a method of facilitating native folding of a recombinant protein in solution.

### RELATED APPLICATIONS

This application claims the benefit of priority to U.S. Provisional Patent Application Ser. No. 60/547,969, filed Feb. 26, 2004; the entirety of which is incorporated by reference.

### BACKGROUND OF THE INVENTION

The process of protein folding is complex, and a complete understanding of it is one of the challenges facing contemporary biochemists. The complexity arises in part from the fact that a nascent protein may not fold into its native state due solely to the influence of the primary solvent (water), but may also interact with other molecules in solution. The effects of other molecules may be favorable for folding, as is the case for molecules like folding chaperones, or unfavorable, as is the case for other partially-unfolded protein molecules.

One of the primary driving forces in protein folding is the burial of exposed hydrophobic residues. Dill, K. A. Biochemistry 1990, 29, 7133-7155. Aggregation results if the hydrophobic collapse occurs in an intermolecular instead of an intramolecular fashion. Because aggregation occurs as a parallel reaction to proper folding, there is kinetic competition between the two pathways. Orsini, G.; Goldberg, VI. E. J. Biol. Chem. 1978, 253, 3453-3458; Zettlmeissl, G.; Rudolph; R.; Jaenicke. R. Biochemistry 1979, 18, 5567-5571; Kiefilaber, T.; Rudolph; R.; Kohler, H.-H.; Buchner, J. Bio/Technology 1991, 9, 825-829; Hevehan, D. L.; Clark, E. D. B. Biotechnol. Bioeng. 1997, 54, 221-230.

Aggregation of misfolded proteins is a significant problem both in vivo and in vitro. Aggregation has been implicated in human diseases, such as Huntington's, Alzheimer's, and Parkinson's Diseases. Taylor, J. P.; Hardy, J.; Fischbeck; K. H. Science 2002, 296, 1991-1995. In applied biotechnology, aggregation is a significant side reaction of protein refolding, which is an important step in the production of many recombinant proteins. De Bemandez Clark, E.; Schwarz, E.; Rudolph, R. Methods Enzymol. 1999, 309, 217-236.

Both nature and man have developed strategies to combat aggregation. Chaperonins, such as the GroEL/GroES system, surround and isolate partially-folded proteins in the bulk cytosol so they can continue to fold without aggregating. Hartl, F. U.; Hayer-Hartl, M. Science 2003, 295, 1852-1858. Similarly, additives to deter aggregation are often included in protein refolding buffers and other in vitro applications, such as pharmaceutical formulations. Wang, W. Int. J. Pharm. 1999, 185, 129-188.

### SUMMARY OF THE INVENTION

Presently disclosed are classes of additives that, when added to protein solutions, attenuate the rate of aggregation. The members of the classes have two key, well-defined properties that result in their ability to slow aggregation. The present invention also recognizes that there are many molecules that exemplify the two properties.

In one embodiment the present invention relates to a compound comprising a non-protein-binding moiety (NPBM) and at least one protein binding group (PBG). In a further embodiment, the NPBM is a polyol, sugar, amino acid, or dendrimer moiety. In a further embodiment, the polyol moiety is a sorbitol or mannitol moiety. In a further embodiment, the sugar moiety is a glucose, sucrose, or trehalose moiety. In a further embodiment, the amino acid moiety is an arginine betaine, proline, or ectoine moiety. In a further embodiment, the dendrimer moiety is based on benzene, pentaerythritol, P(CH₂OH)₃, or TRIS.

In a further embodiment, the PBG is a urea, guanidinium ion, detergent, amino acid, denaturant, surfactant, polysorbate, polaxamer, citrate, chaotrope, or acetate group. In a further embodiment, the PBG is a guanidinium ion. In a further embodiment, the PBG is sodium dodecyl sulfate.

In another embodiment, the present invention relates to a compound of formula I:

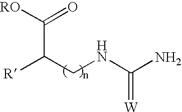

### I

wherein:

R is an electron pair, H, alkyl, aryl, heteroaryl, aralkyl, heteroaralkyl, or an alkali metal;

R′ is H, alkyl, aryl, heteroaryl, aralkyl, heteroaralkyl, or (R″)₃N;

R″ is an electron pair, H, alkyl, aryl, heteroaryl, aralkyl, or heteroaralkyl;

W is O, NH₂⁺, (halogen)⁻, or S; and

n is 1, 2, or 4-100.

In a further embodiment, the present invention relates to a compound of formula I and the attendant definitions, wherein R is an electron pair. In a further embodiment, R′ is H. In a further embodiment, R′ is (R″)₃N. In a further embodiment, R′ is H₃N⁺, . In a further embodiment, W is NH₂⁺, Cl⁻. In a further embodiment, n is 1. In a further embodiment, n is 2. In a further embodiment, n is 4. In a further embodiment, n is 5. In a further embodiment, n is 6. In a further embodiment, R is an electron pair, R′ is H₃N⁺, , W is NH₂+Cl⁻, and n is 1. In a further embodiment, R is an electron pair, R′ is H₃N⁺, , W is NH₂+Cl⁻, and n is 2. In a further embodiment, R is an electron pair, R′ is H₃N⁺, , W is NH₂+Cl⁻, and n is 4. In a further embodiment, R is an electron pair, R′ is H₃N⁺, , W is NH₂+Cl⁻, and n is 5. In a further embodiment, R is an electron pair, R′ is H₃N⁺, , W is NH₂+Cl⁻, and n is 6. In a further embodiment, R is an electron pair, R′ is H₃N⁺, , W is O, and n is 1. In a further embodiment, R is an electron pair, R′ is H₃N⁺, , W is O, and n is 2. In a further embodiment, R′ is H₃N⁺, W is O, and n is 4. In a further embodiment, R is an electron pair, R′ is H₃N⁺, W is O, and n is 5. In a further embodiment, R is an electron pair, R′ is H₃N⁺, ; W is O, and n is 6. In a further embodiment, R is an electron pair, R′ is H, W is NH₂+Cl⁻, and n is 1. In a further embodiment, R is an electron pair, R′ is H, W is NH₂+Cl⁻, and n is 2. In a further embodiment, R is an electron pair, R′ is H⁺, , W is NH₂+Cl⁻, and n is 4. In a further embodiment, R is an electron pair, R′ is H, W is NH₂+Cl⁻, and n is 5. In a further embodiment, R is an electron pair, R′ is H, W is NH₂+Cl⁻, and n is 6. In a further embodiment, R is an electron pair, R′ is H, W is O, and n is 1. In a further embodiment, R is an electron pair, R′ is H, W is O, and n is 2. In a further embodiment, R is an electron pair, R′ is H, W is O, and n is 4. In a further embodiment, R is an electron pair, R′ is H, W is O, and n is 5. In a further embodiment, R is an electron pair, R′ is H, W is O, and n is 6.

In another embodiment, the present invention relates to one of the following compounds:

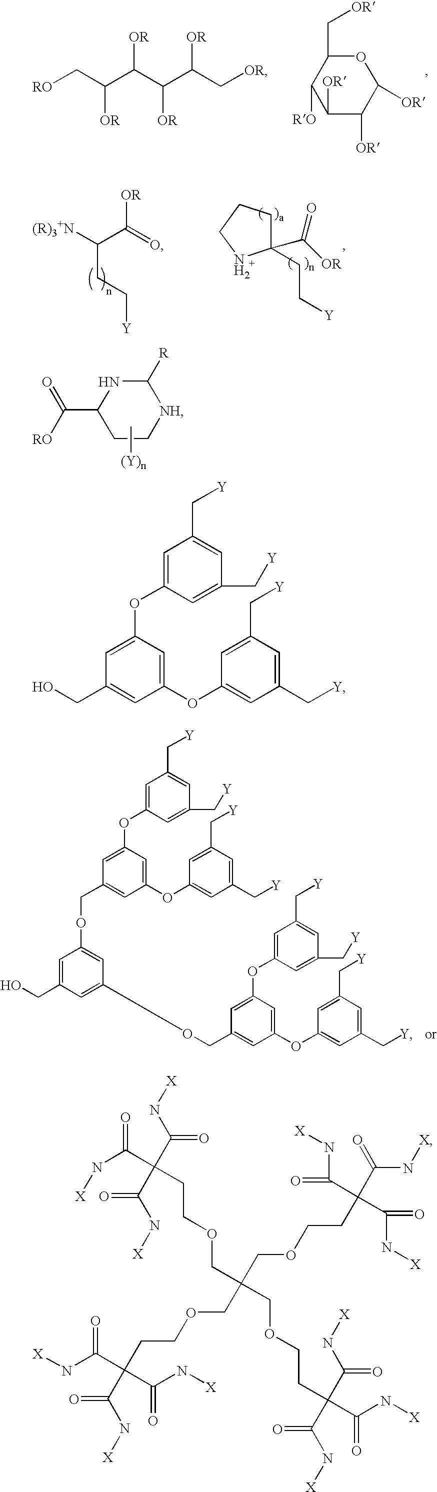

wherein, independently for each occurrence,

R is H or CH₂Y;

R′ is H, a sugar radical, or CH₂Y;

n is an integer from 1 to 100, inclusive;

a is 1, 2, or 3;

X is C(CH₂Y)₃; and

Y is a protein binding group,

wherein at least one Y is present in all compounds.

In a further embodiment, Y is a guanidinium ion.

In another embodiment, the present invention relates to a polymer of formula II, III, IV, V, VI, VII, VIII, or IX:

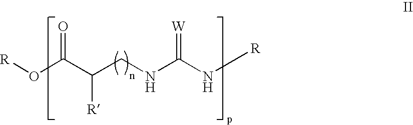

wherein, independently for each occurrence:

R is an electron pair, H, alkyl, aryl, heteroaryl, aralkyl, heteroaralkyl, or an alkali metal;

R′ is H, alkyl, aryl, heteroaryl, aralkyl, heteroaralkyl, or (R″)₃N;

R″ is an electron pair, H, alklyl, aryl, heteroaryl, aralkyl, or heteroaralkyl;

W is O, NH₂⁺, (halogen)⁻, or S;

n is 1, 2, or 4-100; and

p is an integer from 2 to 1000 inclusive;

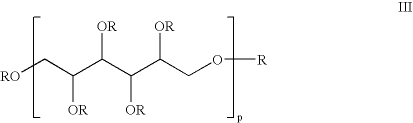

wherein, independently for each occurrence,

R is H, alkyl, aryl, heteroaryl, aralkyl, heteroaralkyl, or an alkali metal, or CH₂Y;

p is an integer from 2 to 1000 inclusive; and

Y is a PBG, wherein at least one Y is present;

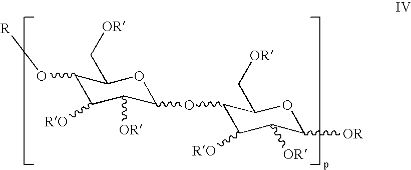

wherein, independently for each occurrence:

R is H, alkyl, aryl, heteroaryl, aralkyl, heteroaralkyl, or an alkali metal, or CH₂Y;

R′ is H, alkyl, aryl, heteroaryl, aralkyl, heteroaralkyl, or (R″)₃N;

R″ is an electron pair, H, alkyl, aryl, heteroaryl, aralkyl, or heteroaralkyl;

p is an integer from 2 to 1000 inclusive; and

Y is a PBG, wherein at least one Y is present;

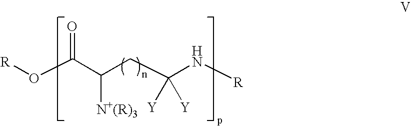

wherein, independently for each occurrence:

R is H, alkyl, aryl, heteroaryl, aralkyl, heteroaralkyl, or an alkali metal, or CH₂Y;

n is an integer from 1 to 100 inclusive;

p is an integer from 2 to 1000 inclusive; and

Y is a PBG;

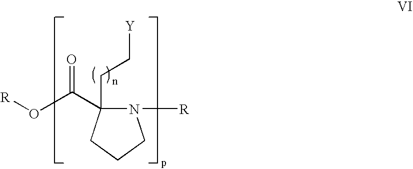

wherein, independently for each occurrence,

R is H, alkyl, aryl, heteroaryl, aralkyl, heteroaralkyl, an alkali metal, or CH₂Y;

n is an integer from 1 to 100, inclusive;

a is 1, 2, or 3;

Y is a PBG; and

p is an integer from 2 to 1000, inclusive;

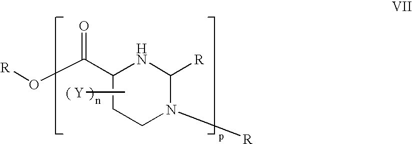

wherein, independently for each occurrence,

R is H, alkyl, aryl, heteroaryl, aralkyl, heteroaralkyl, an alkali metal, or CH₂Y;

n is an integer from 1 to 6, inclusive;

Y is a PBG; and

p is an integer from 2 to 1000, inclusive;

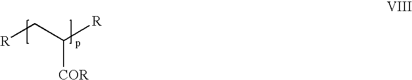

wherein, independently for each occurrence,

R is H, OH, alkyl, alkoxy, aryl, heteroaryl, aralkyl, heteroaralkyl, —O-alkali metal, CH₂Y, OCH₂Y, or has a structure selected from the following:

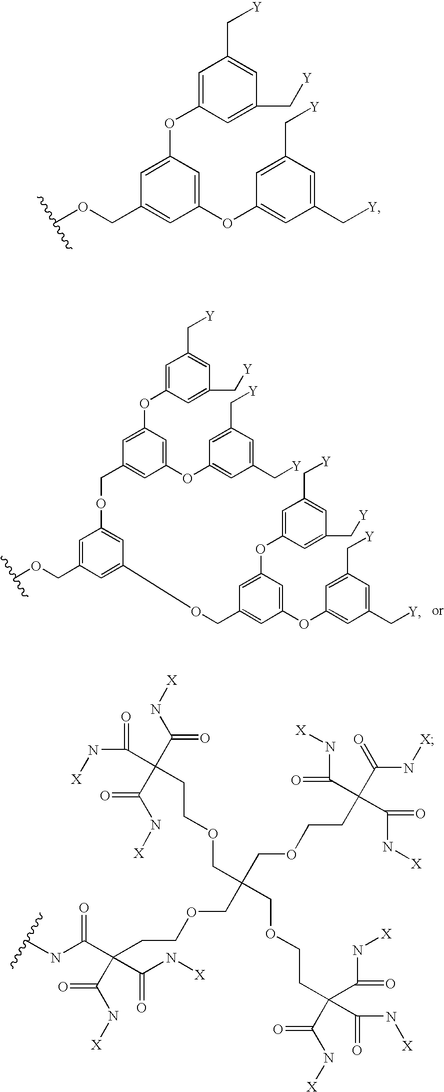

a is 1, 2,or 3;

X is C(CH₂Y)₃;

Y is a PBG, wherein at least one Y is present; and

p is an integer from 2 to 1000, inclusive; or

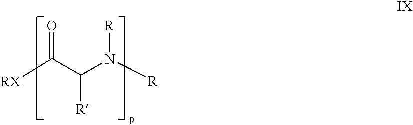

wherein, individually for each occurrence:

R is an electron pair, H, alkyl, aryl, heteroaryl, aralkyl, heteroaralkyl, or an alkali metal;

R′ is a side chain of an alpha-amino acid, wherein at least one instance of R′ is the side chain of arginine;

X is O or NR; and

p is an integer from 2 to 1000, inclusive.

In another embodiment, the present invention relates to a method of screening compounds or polymers for the property of inhibiting protein aggregation in solution, comprising:

a) computing a set of parameters utilizing molecular modeling based on compounds or polymers known to have the property of inhibiting protein aggregation;

b) applying those parameters to other compounds or polymers; and

c) choosing the compounds or polymers that meet the criteria of those parameters.

In another embodiment, the present invention relates to a method of preparing new compounds or polymers having the property of protein aggregation inhibition in solution, comprising:

a) computing a set of parameters utilizing molecular modeling based on compounds or polymers known to have the property of inhibiting protein aggregation;

b) designing compounds or polymers based on those parameters; and

c) synthesizing the compounds or polymers.

In another embodiment, the present invention relates to a method of classifying additives as either inhibitory of protein aggregation in solution or not inhibitory of protein aggregation in solution, comprising:

a) determining the phase space trajectories of the protein, solvent, and additive using molecular dynamics;

b) calculating the distance, r, between the center of mass for both the solvent molecule and additive molecule to the protein's van der Waals surface;

c) determining the minimum distance, r*, at which no significant differences between the local (r=r*) and bulk density are observed;

d) determining which molecules lie within the distance, r*, from the protein surface and classifying these molecules as the local domain;

e) determining which molecules lie outside the distance, r*, from the protein surface and classifying these molecules as the bulk domain;

f) determining the instantaneous preferential binding coefficient, Γ_{XP}(t), using the following formula:

Γ_{XP}(t)=n^{II}ₓ−n^{I}ₓ(n^{II}w/n^{I}_{W})

wherein:

n^{II}ₓ=the number of additive molecules in the bulk domain;

n^{I}ₓ=the number of additive molecules in the local domain;

n^{II}_{w}=the number of solvent molecules in the bulk domain; and

n^{I}_{w}=the number of solvent molecules in the local domain; and

g) calculating the preferential binding coefficient, Γ_{XP}, as the time average of each of the values in step f) using the following formula:

$${Γ}_{XP} = \frac{1}{t} {\int }_{0}^{t} {Γ}_{XP} ( {t}^{′} ) d {t}^{′} .$$

In another embodiment, the present invention relates to a method of suppressing or preventing aggregation of a protein in solution, comprising the step of combining in a solution a compound or polymer of the present invention and a protein.

In a further embodiment, the protein is a recombinant protein. In a further embodiment, the protein is a recombinant antibody. In a further embodiment, the protein is a recombinant human antibody. In a farther embodiment, the protein is a recombinant human protein. In a farther embodiment, the protein is recombinant human insulin, recombinant human erythropoietin or a recombinant human interferon. In a further embodiment, the solution is an aqueous solution. In a farther embodiment, the protein is a recombinant protein; and the solution is an aqueous solution. In a further embodiment, the protein is a recombinant human protein; and the solution is an aqueous solution.

In another embodiment, the present invention relates to a method of decreasing the toxicological risk associated with administering a protein to a mammal in need thereof, comprising the steps of adding to a first solution of a protein a compound or polymer of the present invention to give a second solution; and administering to a mammal in need thereof a therapeutic amount of said second solution.

In a further embodiment, the protein is a recombinant protein. In a further embodiment, the protein is a recombinant antibody. In a farther embodiment, the protein is a recombinant human antibody. In a further embodiment, the protein is a recombinant mammalian protein. In a further embodiment, the protein is a recombinant human protein. In a further embodiment, the protein is recombinant human insulin, recombinant human erythropoietin or a recombinant human interferon. In a further embodiment, the first solution and the second solution are aqueous solutions. In a further embodiment, the protein is a recombinant protein; and the first solution and the second solution are aqueous solutions. In a further embodiment, the protein is a recombinant human antibody; and the first solution and the second solution are aqueous solutions. In a further embodiment, the protein is a recombinant human protein; and the first solution and the second solution are aqueous solutions.

In another embodiment, the present invention relates to a method of facilitating native folding of a recombinant protein in solution, comprising the step of combining in a solution a compound or polymer of the present invention and a recombinant protein.

In a further embodiment, the recombinant protein is a recombinant antibody. In a further embodiment, the recombinant protein is a recombinant human antibody. In a further embodiment, the recombinant protein is a recombinant mammalian protein. In a further embodiment, the recombinant protein is a recombinant human protein. In a further embodiment, the recombinant protein is recombinant human insulin, recombinant human erythropoietin or a recombinant human interferon. In a further embodiment, the solution is an aqueous solution. In a further embodiment, the recombinant protein is a recombinant human antibody; and the solution is an aqueous solution. In a further embodiment, the recombinant protein is a recombinant human protein; and the solution is an aqueous solution.

These embodiments of the present invention, other embodiments, and their features and characteristics, will be apparent from the description, drawings and claims that follow.

### BRIEF DESCRIPTION OF THE FIGURES

FIG. 1 depicts a simplified dimerization reaction-coordinate diagram for the reaction U+U A₂ (equation 2). The dotted line is the reaction coordinate in water and the solid line is the reaction coordinate in the presence of an additive having the two anti-aggregation properties discussed. Protein molecules are represented by black coils and the additive by dark grey circles. The energy difference between the reactants (U+U) and the transition state determines the rate of the reaction. In the A₂ state, the region between the protein molecules (light grey oval) is preferentially hydrated because water can enter this region but the additive cannot. This preferential hydration increases the free energy of the transition state, increases the energy barrier for the reaction, and slows the reaction rate.

FIG. 2 depicts arginine derivatives with shorter (left) and longer (right) methylene linkers between their amino acid backbone and guanidino functional groups.

FIG. 3 depicts molecules that will be preferentially-oriented at the protein-solvent interface. Molecule (a) is a derivative of glucose (stabilizer) linked to a dimethyl-guanidino (destabilizer) moiety. Molecule (b) is a polyol (stabilizer) with a guanidino group (destabilizer) attached to one end.

FIG. 4 depicts the physical interpretation of the preferential binding coefficient. Interactions of solvent molecules with the protein at the protein-solvent interface generally induce solvent concentration differences in the local (II) and bulk (I) domains. Γ_{XP} is the thermodynamic measure of the number of additive molecules bound to the protein, or in other words, the excess number of additive molecules in the vicinity of the protein versus the number of additive molecules in an equivalent volume of bulk solution.

FIG. 5 depicts a simulation cell containing RNase T1 (center spheres) solvated by water (thin lines) and urea (spheres).

FIG. 6 depicts radial distribution functions of water, urea, and glycerol shown for simulations of RNase T1 in glycerol and urea solutions (left) and RNase A in a glycerol solution (right). In the left-hand figure, the difference between the two gw(r) functions is not visible at this scale.

FIG. 7 depicts apparent preferential binding coefficient as a function of the cutoff distance between the local and bulk domains for simulations of RNase T1 in glycerol and urea solution.

FIG. 8 depicts Γₓₚ(t) probability density function. A wide range of values of Γₓₚ(t) are sampled as water and cosolvent molecules diffuse between the local and bulk domains.

FIG. 9 depicts the correlation of solvent-accessible area and the number of water molecules in the local domain of constituent groups. Each point represents a constituent group of either a type of amino acid side chain or the protein backbone in one of the three simulations shown in Table 2. The solvent accessible area of a constituent group and the number of water molecules in the local domain of the solvent near the group (n_{wi}) are correlated.

FIG. 10 depicts the binding behavior of glycerol and water with the 15 serine residues in RNase T1 as shown in a plot of the number of glycerol molecules in the local domain of each serine residue versus the number of water molecules in the same volume. The labels are the one-letter codes for each amino acid side chain, and “B” is the protein backbone. The line represents the bulk glycerol composition. Ser 17, 35, and 72 have positive preferential binding coefficients, Ser 63 has a negative preferential binding coefficient, and the remaining 11 serine residues have essentially zero values for their preferential binding coefficients.

FIG. 11 depicts the local binding behavior of urea and water with the amino acid backbone and side chains in RNase T1. The labels are the one-letter codes for the amino acid side chains, and “B” is the protein backbone. The line denotes the bulk urea concentration. In addition to the protein backbone and Ser, the hydrophobic amino acids Cys, Gly, Leu, Phe, Pro, Tyr, and Val all preferentially bind urea, while the hydrophilic Asp preferentially binds water.

FIG. 12 depicts the group preferential binding coefficients for glycerol with the amino acid backbone and side chains in RNase T1. The labels are the one-letter codes for the amino acid side chains, and “B” is the protein backbone. The line denotes the bulk glycerol concentration. Tyr and Gly preferentially bind glycerol; Asp and Glu preferentially bind water; and the binding coefficients of the other groups are not statistically different from zero.

FIG. 13 depicts the local binding behavior of glycerol with the amino acid backbone and side chains in RNase A. The labels are the one-letter codes for the amino acid side chains, and “B” is the protein backbone. The line denotes the bulk glycerol concentration. All of the constituent groups in RNase A either preferentially bind water or are neutral.

FIG. 14 depicts the Biacore 3000 surface plasmon resonance data for insulin binding to immobilized anti-insulin. Raw binding data (solid curves) are shown with a three-parameter, least squares fit to all the data (dashed curves). The detector response is proportional to the mass of antigen bound to the antibody immobilized in the flow cell.

FIG. 15 depicts the calculated free energies for a pair of 20 Å spherical proteins into 1M arginine and guanidinium solutions as a function of the separation between the proteins. Free energies are normalized to the free energy of the dissociated pair (x>10 Å). The gray spheres indicate the geometry of the protein pair as a function of protein separation. The table shows the magnitudes of the changes in the association and dissociation rate constants (ka and kd).

FIG. 16 depicts the effect of refolding buffer composition on carbonic anhydrase refolding yield. The points are experimental esterase activity data, and the lines are the best fit to a one-parameter, first versus second order kinetic model (equation 32).

### DETAILED DESCRIPTION OF THE INVENTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

The term “additive” as used herein refers to any component other than the subject protein and the main solvent. Non-limiting examples of additives include small molecules, cosolvents, buffer salts, and stabilizers.

The term “dendrimer” is used to mean a broad class of polymers constructed via stepwise polymerization from a central “core unit,” one or more “branching units,” and several “surface units.” The review of Matthews (1998) provides an overview of dendrimers including compositions and synthetic routes. Core units may include (but are not limited to) carbon, nitrogen, phosphorous, benzene, and porphyrins. A non-extensive collection of 17 specific chemistries that are used to create branching units are summarized in Table 2 of Matthews (1998).

The term “TRIS” is art-recognized and refers to tris(hydroxymethyl)aminomethane.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chuomene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulffhydryl, imino, amido, phosphonate, phosphinate, carbonyl, carboxyl, silyl, ether, allkylthio, sulfonyl, ketone, aldehyde, ester, a heterocyclyl, an aromatic or hetero aromatic moiety, —CF₃, —CN, or the like.

The term “carbocycle” is art-recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and include both unsubstituted and substituted amines, e.g., a moiety that may be represented by the general formulas:

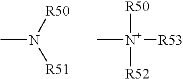

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

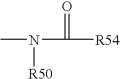

wherein R50 is as defined above, and R54 represents a hydrogen, an alkyl, an alkenyl or —(CH₂)ₘ—R61, where m and R61 are as defined above.

The term “amido” is art-recognized as an amino-substituted carbonyl and includes a moiety that may be represented by the general formula:

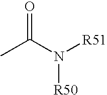

wherein R50 and R51 are as defined above. Certain embodiments of the amide in the present invention will not include imides which may be unstable.

The term “alkylthio” is art-recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art-recognized and includes such moieties as may be represented by the general formulas:

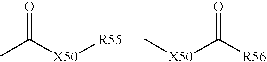

wherein X50 is a bond or represents an oxygen or a sulfur, and R55 represents a hydrogen, an alkyl, an alkenyl, —(CH₂)_{m—R}61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art-recognized and includes a moiety that may be represented by the general formula:

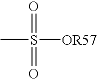

in which R57 is an electron pair, hydrogen, alkyl, cycloalkyl, or aryl.

The term “sulfate” is art-recoginized and includes a moiety that may be represented by the general formula:

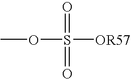

in which R57 is as defined above.

The term “sulfonamido” is art-recognized and includes a moiety that may be represented by the general formula:

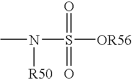

in which R50 and R56 are as defined above.

The term “sulfamoyl” is art-recognized and includes a moiety that may be represented by the general formula:

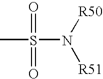

in which R50 and R51 are as defined above.

The term “sulfonyl” is art-recognized and includes a moiety that may be represented by the general formula:

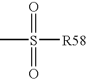

in which R58 is one of the following: hydrogen, alkyl, alkenyl, alkynyl, cycloalkyl, heterocyclyl, aryl or heteroaryl.

The term “sulfoxido” is art-recognized and includes a moiety that may be represented by the general formula:

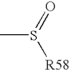

in which R58 is defined above.

The term “phosphoramidite” is art-recognized and includes moieties represented by the general formulas:

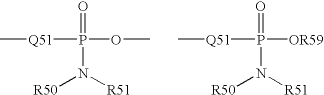

wherein Q51, R50, R51 and R59 are as defined above.

The term “phosphonamidite” is art-recognized and includes moieties represented by the general formulas:

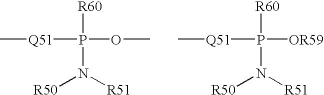

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

For purposes of this invention, the chemical elements are identified in accordance with the Periodic Table of the Elements, CAS version, Handbook of Chemistry and Physics, 67th Ed., 1986-87, inside cover.

### Overview

Proteins are widely used in medical and industrial applications. One of the major difficulties encountered in these applications is that proteins are prone to degradation by a variety of routes, the most common of which is aggregation. Aggregation is the assembly of non-native protein conformations into multimeric states, often leading to phase separation and precipitation. Aggregated protein generally does not have the same functionality as normal, native protein. The problem of aggregation is especially grave in the pharmaceutical industry and in biotechnology, where it can be necessary to handle and store proteins at high concentrations and temperatures and for long periods of time. For example, in pharmaceutical applications, the consequences of administering aggregated drug to a patient can be severe because aggregates can be cytotoxic; and they generally induce an immune response. Bucciatini, M.; Giannoni, E.; Chiti, F.; Baroni, F.; Formigh, L.; Zurdo, J.; Taddei, N.; Ramponi, G.; Dobson, C. M.; Stefani, M. Nature 2002, 416, 507-511; Braun, A.; Kwee, L.; Labow, M. A.; Alsenz, J. Pharm. Res. 1997, 14, 1472-1478. Due to these and other negative effects, protein solutions often contain one or more additives designed to deter aggregation. Wang, W. Int. J. Pharm. 1999, 185,129-188. In addition to aggregation being important in the storage of proteins, it is the dominant mode of protein degradation in protein refolding. Overproduction of recombinant proteins often results in a majority of the protein being produced in the form of phase-separated inclusion bodies. Lilie, H., Schwarz, E., & Rudolph, R. (1998) Curr. Opin. Biotech. 9, 497-501. When this occurs, the inclusion bodies must be harvested, solubilized with a strong denaturant, and then refolded by removal of the denaturant to yield active protein. When the denaturant is removed, the hydrophobic effect drives the unfolded protein molecules to sequester their hydrophobic groups. Dill, K. A. (1990) Biochemistry 29, 7133-7155.

This can occur either in an intramolecular fashion (proper protein folding) or an intermolecular fashion (aggregation), as illustrated schematically by the following reactions:

U→N (1)

U+U→A₂ (2)

where U represents an unfolded protein; N represents a folded, native protein; and A₂ represents a small aggregate species. Thus, there is direct competition between proper protein refolding and aggregation. Zetthneissl, G., Rudolph, R., & Jaenicke, R. (1979) Biochemistry 18, 5567-5571.

Alternatively, if the protein is initially in its native state, such as in a pharmaceutical formulation, aggregation proceeds through formation of a partially-unfolded intermediate, I, which can aggregate in a sense analogous to an unfolded protein:

N [symbol] I (3)

I+I→A₂ (4)

For industrial and medical applications, it is desirable to eliminate or minimize the formation of protein aggregates. In protein folding or refolding processes, decreasing the rate of aggregation results in a higher yield of active, properly-folded protein. In pharmaceutical formulations, decreasing the rate of aggregation causes more drug to remain in its active form and eliminates the possibly dangerous side effects of administering aggregated protein to the patient. To minimize aggregation, various conditions, such as temperature, pH, and the type and amount of buffer additives, are screened experimentally to identify an optimum set of conditions.

Empirically, it has been observed that by adding low molecular weight components, such as salts, sugars, or polyols, to protein solutions, the propensity of a protein to aggregate can often be affected significantly. Wang, W. (1999) Int. J. Pharm. 185, 129-188; Cleland, J. L., Powell, M. F., & Shire, S. J. (1993) Crit. Rev. Ther. Drug Carrier Systems 10, 307-377. Unfortunately, because proteins are diverse in chemistry and structure, additives that work well for a particular protein may not work universally. In addition, current understanding of the mechanisms by which additives confer stability on proteins is limited. Thus, there is often no theoretical guidance to aid in selection of optimal additives, necessitating that protein stabilization be carried out on a case-by-case basis using heuristic experimental screens. This gap in understanding has prevented development of rational strategies to prevent protein aggregation.

Through the mechanistic understanding summarized presently, two fundamental properties of a good anti-aggregation additive have been identified. This discovery allows additives to be selected based on their relative ranking in terms of these two properties, thus narrowing experimental testing to molecules likely to have optimal performance. It also enables molecules to be classified based on whether they may have the ability to attenuate aggregation. The rational, mechanistic classification schemes of the present invention will allow entire classes of protein-aggregation-attenuating additives and formulations to be identified.

Additionally, a quantitative method based on molecular dynamics simulations using all atom potential models has been developed and validated for calculating preferential binding coefficients. The present invention is not a derivative of thermodynamic integration or thermodynamic perturbation methods and requires only a single trajectory to compute the transfer free energy of a protein into a weak-binding additive system. The results match experimental data well for glycerol and urea solutions, covering a range of positive and negative binding behavior. The present invention also augments experimentally-observable, macroscopic thermodynamics with the mechanistic insight provided by a molecular-level, statistical mechanical model.

Variations in the radial distribution functions with distance for each additive are evident up to about 6 Å, i.e., roughly two solvation shells of water, away from the protein. Glycerol is not totally excluded from close contact with the protein, but glycerol is less likely than urea to be found in such a position. The radial distribution functions of water and additives are sufficient to calculate preferential binding coefficients by integrating over a suitable solvent volume.

The binding behavior of the amino acid side chains in RNase T1 qualitatively follow a hydrophilic series, with more hydrophilic amino acids in the protein tending to have a higher concentration of water in their vicinity. The constituent group binding behavior differs between the groups in RNase A to those in RNase T1. Development of a group contribution method at the amino acid level for estimating binding coefficients or transfer free energies of whole proteins is complicated by the wide range of coordination behaviors observed for single types of amino acids in different environments on the protein surface.

In the pharmaceutical industry, many protein drugs are synthesized in bacterial hosts, such as E. coli, in the form of solid, partially-aggregated precipitates called inclusion bodies. These inclusion bodies must be unfolded and solubilized, and then refolded to form active protein. During refolding, proteins are especially susceptible to aggregation, and additives must be used to minimize aggregation and increase the yield of biologically-active protein. The compounds of the present invention are ideal for use in these circumstances because they will slow the rate of aggregation and therefore increase the yield of active protein. Likewise, when pharmaceutically-active proteins are formulated in aqueous solution, additives are used to prevent aggregation during storage, thereby increasing its shelf-life. The compounds of the present invention are also useful in preventing aggregation in these circumstances. Additional applications can be envisioned by those of ordinary skill in the art of protein stabilization. The above applications are meant to be only exemplary and not limiting in any way.

### Select Preferred Embodiments

In a preferred embodiment, the present invention relates to a method of suppressing or preventing aggregation of a protein in solution, comprising the step of combining in a solution a compound of the present invention and a protein. In certain embodiments, the protein is a recombinant protein. In certain embodiments, the protein is a recombinant antibody. In certain embodiments, the protein is a recombinant human antibody. In certain embodiments, the protein is a recombinant mammalian protein. In certain embodiments, the protein is a recombinant human protein. In certain embodiments, the protein is recombinant human insulin, recombinant human erythropoietin or a recombinant human interferon. In certain embodiments, the solution is an aqueous solution. In certain embodiments, the protein is a recombinant protein; and the solution is an aqueous solution. In certain embodiments, the protein is a recombinant human antibody; and the solution is an aqueous solution. In certain embodiments, the protein is a recombinant human protein; and the solution is an aqueous solution.

In a preferred embodiment, the present invention relates to a method of suppressing or preventing aggregation of a protein in solution, comprising the step of combining in a solution a compound of the present invention and a protein. In certain embodiments, the protein is a recombinant protein. In certain embodiments, the protein is a recombinant antibody. In certain embodiments, the protein is a recombinant human antibody. In certain embodiments, the protein is a recombinant mammalian protein. In certain embodiments, the protein is a recombinant human protein. In certain embodiments, the protein is recombinant human insulin, recombinant human erythropoietin or a recombinant human interferon. In certain embodiments, the solution is an aqueous solution. In certain embodiments, the protein is a recombinant protein; and the solution is an aqueous solution. In certain embodiments, the protein is a recombinant human antibody; and the solution is an aqueous solution. In certain embodiments, the protein is a recombinant human protein; and the solution is an aqueous solution.

In a third preferred embodiment, the present invention relates to a method of decreasing the toxicological risk associated with administering a protein to a mammal in need thereof, comprising the steps of adding to a first solution of a protein a compound of the present invention to give a second solution; and administering to a mammal in need thereof a therapeutic amount of said second solution. In certain embodiments, the protein is a recombinant protein. In certain embodiments, the protein is a recombinant antibody. In certain embodiments, the protein is a recombinant human antibody. In certain embodiments, the protein is a recombinant mammalian protein. In certain embodiments, the protein is a recombinant human protein. In certain embodiments, the protein is recombinant human insulin, recombinant human erythropoietin or a recombinant human interferon. In certain embodiments, the first solution and the second solution are aqueous solutions. In certain embodiments, the protein is a recombinant protein; and the first solution and the second solution are aqueous solutions. In certain embodiments, the protein is a recombinant human antibody; and the first solution and the second solution are aqueous solutions. In certain embodiments, the protein is a recombinant human protein; and the first solution and the second solution are aqueous solutions.

In another preferred embodiment, the present invention relates to a method of facilitating native folding of a recombinant protein in solution, comprising the step of combining in a solution a compound of the present invention and a recombinant protein. In certain embodiments, the recombinant protein is a recombinant antibody. In certain embodiments, the recombinant protein is a recombinant human antibody. In certain embodiments, the recombinant protein is a recombinant mammalian protein. In certain embodiments, the recombinant protein is a recombinant human protein. In certain embodiments, the recombinant protein is recombinant human insulin, recombinant human erythropoietin or a recombinant human interferon. In certain embodiments, the solution is an aqueous solution. In certain embodiments, the recombinant protein is a recombinant human antibody; and the solution is an aqueous solution. In certain embodiments, the recombinant protein is a recombinant human protein; and the solution is an aqueous solution.

### Kinetic Model Approach for Stabilizing Proteins Towards Aggregation

To see how additives affect aggregation rate, the rate constant for aggregation, k_{agg}, can be expressed using transition state theory as:

$${k}_{agg} = \frac{{k}_{b} T}{h} {K }^{‡} \qquad ( 5 )$$

where k_{b} is Boltzmann's constant, T is the absolute temperature, h, is Planck's constant, and K⁵⁵⁵ is the equilibrium constant between the reactants and the transition state for the reaction (either equation 2 or 4). The change in relative reaction rate due to an additive (X) at constant temperature and pressure can therefore be expressed as:

$${( \frac{\partial ln {k}_{agg}}{\partial {m}_{x}} )}_{T , P , {m}_{P}} = {( \frac{\partial ln {K}^{‡}}{\partial {m}_{x}} )}_{T , P , {m}_{P}} \qquad ( 6 )$$

where mₓ is the morality of additive. Using the Wyman linkage relation, the above expression can be written in terms of the extent of binding of the additive to the protein species:

$$\begin{matrix}{( \frac{\partial ln {k}_{agg}}{\partial {m}_{x}} )}_{T , P , {m}_{P}} = {( \frac{\partial ln {a}_{x}}{\partial {m}_{x}} )}_{T , P , {m}_{P}} {( \frac{\partial ln {k}_{agg}}{\partial ln {a}_{x}} )}_{T , P , {m}_{P}} \\ = {( \frac{\partial ln {a}_{x}}{\partial {m}_{x}} )}_{T , P , {m}_{P}} ( {Γ}_{XP}^{‡} - {Γ}_{XP}^{R} )\end{matrix} \qquad ( 7 ) , ( 8 )$$

where aₓ is the thermodynamic activity of additive, and each Σ is a preferential binding coefficient. Wyman Jr., J. Adv. Protein Chem. 1964, 19, 223-286; Timasheff, S. N. PNAS 2002, 99, 9721-9726; Baynes, B. M.; Trout, B. L. J. Phys. Chem. B 2003, submitted for publication. Σ^{‡}_{PX} is the number of additive molecules bound to the transition state of equation 2 or 4, and Σ^{R}_{PX} is the number of additive molecules bound to the reactant in the same equation. Since (∝ln a_{X}/∝m_{X})_{T,P,mp} is positive, equation 8 shows that in order for an additive to decrease the rate of aggregation, the additive must bind less to the transition state than to the reactant, making Σ^{‡}_{XP}−Σ^{R}_{XP} negative.

### Attenuation of Protein Aggregation

In the pharmaceutical industry today, a refolding buffer additive used to increase the yield of active protein is the amino acid L-arginine. Arginine has very little effect on the folding equilibrium yet it facilitates refolding of several type of proteins from the unfolded state, such as tPA, interferon γ, lysozyme, carbonic anhydrase B, factor XIII, and antibodies. Arakawa, T. & Tsumoto, K. (2003) Biochem. Biophys. Res. Comm. 304, 148-152; Taneja, S. & Ahmad, F. (1994) Biochem. J. 303, 147-153; Shiraki, K., Kudou, M., Fujiwara, S., Innanaka, T., & Takagi, M. (2002) J. Biochem. 132, 591-595; Rudolph, R.; Fischer, S.; Mattes, R. 1985; Arora, D.; Khanna, N. J Biotechnol. 1996, 52, 127-133; Armstrong, N.; de Lencastre, A.; Gouaux, E. Protein Sci. 1999, 8, 1475-1483; Rinas, U.; Risse, B.; Jaenicke, R.; Abel, K. J., Zettleneissl, G. Biol. Chem. Hoppe-Seyler 1990, 371, 49-56; Buchner, J.; Rudolph, R. Biotechnology 1991, 9, 157-162. Arginine has been shown to increase the yield of renatured protein by decreasing the rate of aggregation. Hevehan, D. L.; Clark, E. D. B. Biotechnol. Bioeng. 1997, 54, 221-230. While a mechanism which can explain how arginine functions has not been proposed, these results suggest that arginine selectively slows protein-protein association (equation 2) while having little effect on protein folding (equation 1). Lilie, H., Schwarz, E., & Rudolph, R. (1998) Curr. Opin. Biotech. 9, 497-501; Tsumoto, K., Umetsu, M., Kumagai, I., Ejima, D., Philo, J. S., & Arakawa, T. (2004) Biotechnol. Prog. 20, 1301-1308.

In recent theoretical studies of the effects of solution additives on protein aggregation and association, a theory was developed that may explain how arginine deters aggregation. Baynes, B. M. & Trout, B. L. (2004) Biophys. J. 87, 1631-1639. This theory builds on previous molecular-level understanding of additive effects on protein thermodynamics, preferential binding, osmotic stress, and Kirkwood-Buff theory. Baynes, B. M. & Trout, B. L. 2003 J. Phys. Chem. B 107, 14058-14067; Timasheff, S. N. (1998) Adv. Protein Chem. 51, 355-431; Colombo, M. F., Rau, D. C., & Parsegian, A. (1992) Science 256, 655-659; Kirkwood, J. G. & Buff, F. P. (1951) J. Chem. Phys. 19, 774-777; Shimizu, S. (2004) PNAS USA 101, 1195-1199; Shimizu, S. & Smith, D. J. (2004) J. Chem. Phys. 121, 1148-1154; Smith, P. E. (2004) J. Phys. Chem. B. 108, 16271-16278.

“Gap effect theory” suggests that solution additives much larger than water which do not affect the free energy of isolated protein molecules will selectively increase the free energy of protein-protein encounter complexes. This effect will increase the activation free energy for association, and therefore slow protein-protein association reactions. The accompanying effect on intramolecular reactions such as refolding is predicted to be small.

It is presently disclosed that arginine has a critical combination of two simple factors that enable it to prevent aggregation during folding. These factors include size and binding.

1. Size. Arginine is a much larger molecule than water, the primary solvent.

2. Binding. Protein molecules in isolation do not have a significant preference to be solvated by either arginine or water.

We termed solution additives that have the above properties “neutral crowders” because of their size (crowder) and affinity for isolated protein molecules (neutral). The effect of such molecules on protein association reactions contrasts with that of excluded or hard-sphere crowders, which can accelerate association, and generally shift the association equilibrium toward the associated state. Minton, A. P. (1997) Curr. Opin. Biotech. 8, 65-69; Linder, R. & Ralston, G. (1995) Biophys. Chem. 57, 15-25.

On the basis of the above theoretical developments and the existing experimental data on arginine systems, it was hypothesized that arginine is a neutral crowder, and it exerts its beneficial effect on protein refolding by slowing protein association reactions with only a small concomitant effect on the rate of protein refolding.

Because gap effect theory predicts that arginine should decrease protein-protein association rates in general, this effect can be tested in any convenient system. Two types of protein association reactions for study were selected: the association of insulin with a monoclonal antibody to insulin (globular protein association) and association of folding intermediates and aggregates of carbonic anhydrase II (aggregation during refolding). By performing these association tests in different buffers, the effect of arginine in the buffer can be deduced by comparison. In parallel, the effects of guanidinium chloride on the same association/aggregation systems was assessed. Finally, the experimental results were reconciled with gap effect theory.

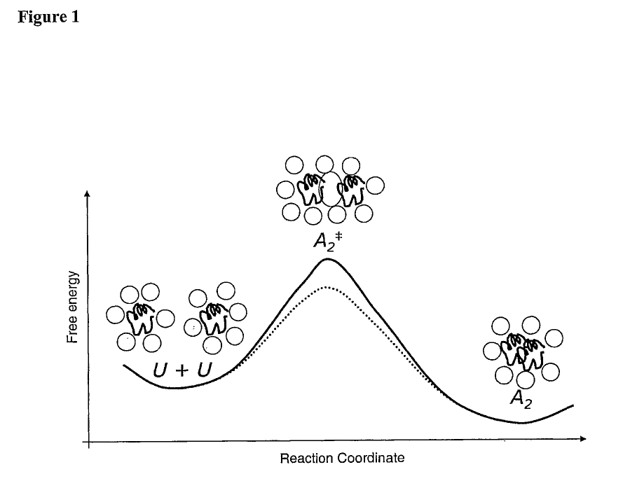

The mechanism by which the factors above affect aggregation is shown schematically in FIG. 1. As the protein molecules diffuse toward each other, the size property ensures that a region of preferential hydration will form between the protein molecules because water but not the additive can fit in the gap (the oval in the transition state A₂⁵⁵⁵ of FIG. 1). This is analogous to “osmotic stress” effects on the equilibrium between two macromolecular conformations where one conformation has a crevice that water can enter but an additive cannot. Parsegian, V. A.; Rand, R. P.; Rav, D. C. PNAS USA 2000, 97, 3987-3992. The binding property ensures that when there is no steric constraint due to such a gap, arginine and water can solvate the protein equally well. This means that the region of preferential hydration shown in FIG. 1 is the only contribution to the preferential binding coefficients of the additive with the protein in any of the three states shown (U+U, A₂^{‡}, A₂). Because the transition state is preferentially hydrated, Γ^{‡}_{XP} is negative. Therefore the quantity Γ^{‡}_{XP}−Γ^{R}_{XP} is negative and aggregation is slowed. Any additive that has these two properties will deter aggregation during folding or in any other situation where a bimolecular step is rate limiting.

The size and binding properties are both necessary for prevention of aggregation. Molecules that meet the size criterion but not the binding criterion will either accelerate aggregation (such as “crowders” like dextran) or be denaturants (such as guanidinium chloride) and therefore have other undesirable effects on protein stability. Linder, R.; Ralston, G. Biophys. Chem. 1995, 57; 15-25; Orsini, G.; Goldberg, M. E. J. Biol. Chem. 1978, 253, 3453-3458; Jasuja, R. Technical Report, Business Communications Company, Inc., 2000. A molecule that does not meet the size criterion but meets the binding criterion will have almost no effect on aggregation.

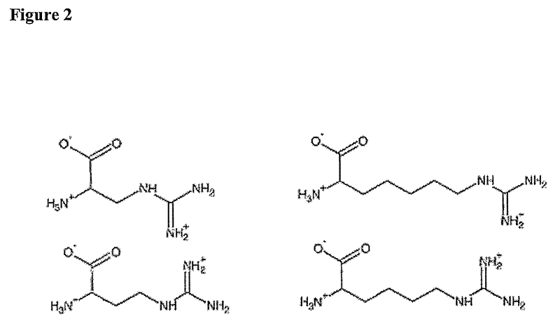

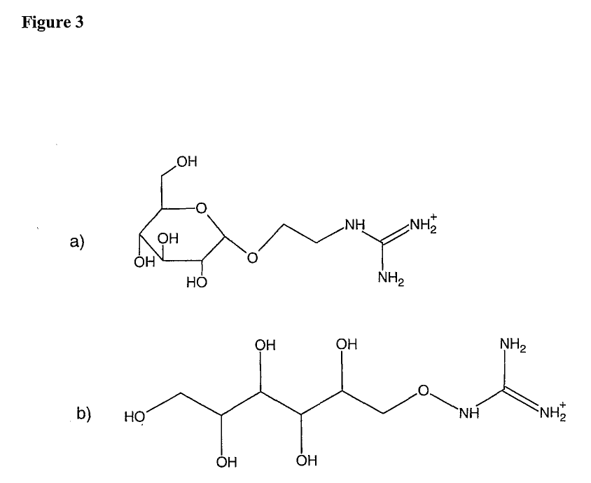

The two properties above differentiate molecules that may have advantageous effects on aggregation via the mechanism above from those that may not. It is believed that there are many molecules that have not been used as additives which have both of the above properties. Since these properties are presently disclosed, arginine was not selected with them in mind, implying that another yet untested molecule may exemplify the properties to a larger extent and have superior aggregation preventing characteristics. As non-limiting examples, some molecules with the two properties above that may prevent aggregation via a similar mechanism include:
Citrulline
Arginine or citrulline derivatives with a longer or shorter methylene linker between the amino acid backbone and guanidino or urea group (FIG. 2).
Arginine or citrulline derivatives where the amino acid backbone group is replaced by another large functional group which does not bind to proteins. (For example, 2-guanidino acetic acid, 3-guanidino propanoic acid, 4-guanidino butyric acid, 5-guanidino pentanoic acid, etc.)
Molecules that are not randomly orientated in solution near proteins. Such molecules can be constructed by covalently attaching a molecule which stabilizes proteins against unfolding with a molecule that destabilizes proteins against unfolding. Examples of novel molecules designed based on this idea are shown in FIG. 3. A partial list of molecules that are known to stabilize and destabilize proteins against unfolding are shown in Table 1.

TABLE 1

| Protein Stabilizer | Protein Destabilizer |
|---|---|
| Sugars (e.g. glucose, sucrose, | Urea |
| trehalose) | |
| Polyols (e.g. sorbitol, mannitol) | Guanidinium chloride |
| Dextran | Detergents (e.g. sodium |
| | dodecyl sulfate, |
| | Tris) |
| Kosmotropes | Chaotropes |
| Glycine, glycine betaine | |

### Compounds and Polymers of the Present Invention

Based on the studies described in the previous section, compounds and polymers of the present invention may be prepared by functionalizing a molecule or monomer that does not bind to a protein with at least one protein binding group. In other words, compounds and polymers of the present invention possess a non protein bonding moiety and a protein binding group. Molecules that do not bind to proteins include but are not limited to osmolytes and kosmotropes, such as glycerol, glycine betaine, dendrimers, and trimethyl amine N-oxide. Other such molecules are known to those skilled in the art.

A protein-binding group is a molecule or functional group that binds to some proteins. Many molecules that fall in this class are, for example, denaturants or surfactants. Some non-limiting examples of protein-binding molecules are: the guanidinium ion, urea, amino acids (such as arginine, lysine, aspartate, glutamate), sodium dodecyl sulfate, tweens (polysorbate), poloxamers, and ions (such as citrate and acetate). A group or molecule does not need to bind to all proteins to be classified as a “protein-binding group;” rather, it merely needs to bind to some proteins. The concepts of “binding” and groups or molecules that bind to proteins are well-known to those skilled in the art.

The net effect of functionalizing a non-binder with a protein-binding group will be to move the protein preferential binding coefficient toward zero. Molecules that are large, but have a protein preferential binding coefficient near zero, have the properties that they prevent aggregation but do not destabilize native protein molecules. Thus, these molecules are useful as anti-aggregation additives.

Polymers of the present invention may be prepared in a number of ways. A monomer may be functionalized to include a protein binding group or both a protein and non protein binding group. Polymerization of the functionalized monomer may be by methods generally known in the art. The non protein binding group and the protein binding group may each be, individually, incorporated within the backbone of the polymer or within a pendant chain of the polymer, or both. In the case of dendrimer or star polymers the two groups may each be, individually, a part of the polymer network or pendant to the polymer network, or both. Another way to prepare the polymers of the present invention includes functionalizing a preformed polymer with a protein binding group or with both a protein binding group and non protein binding group. For example, it is envisioned by the inventors that one may start with a polyacrylic acid and saponify the acid groups to introduce a protein binding group or both a protein and non-protein binding group.

### Statistical Model Approach for Stabilizing Proteins Towards Aggregation

Additives perturb the chemical potential of the protein system by associating either more strongly or more weakly with the protein than water. This phenomenon, called “preferential binding,” is of great interest because it governs the physical and chemical properties of proteins. Timasheff; S. N. Adv. Protein Chem. 1998, 51, 355-431.

When an additive (X) is added to an aqueous protein solution, it alters the chemical potential of the protein (μp) via the following relationship:

$$\begin{matrix}Δ {μ}_{P}^{tr} = {\int }_{0}^{{m}_{X}} {( \frac{\partial {μ}_{P}}{\partial {m}_{X}} )}_{{m}_{P}} d {m}_{X} \\ = - {\int }_{0}^{{m}_{X}} {( \frac{\partial {μ}_{X}}{\partial {m}_{X}} )}_{{m}_{P}} {( \frac{\partial {m}_{X}}{\partial {m}_{P}} )}_{{μ}_{X}} d {m}_{X}\end{matrix} \qquad ( 9 ) , ( 10 )$$

where Δμp is the transfer free energy of the protein from pure water into the mixed solvent system, in is molality, and subscripts X and P identify the additive and protein respectively. Lee, J. C.; Timasheff, S. N. J. Biol. Chem. 1981, 256, 7193-7201. Two partial derivatives appear in equation 10. The first captures the dependence of the additive chemical potential on additive molality and can be evaluated by experiments on a binary mixture of additive and water (mp→0). The second partial derivative is the “preferential binding coefficient;” Γ_{XP}:

$${Γ}_{XP} = {( \frac{\partial {m}_{X}}{\partial {m}_{P}} )}_{PX} \qquad ( 11 )$$

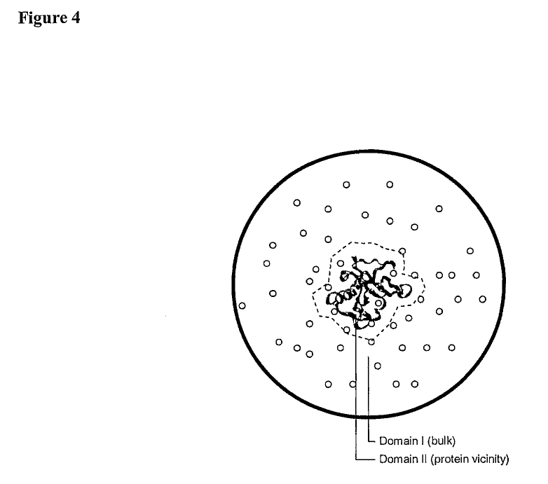

The preferential binding coefficient is a way in which binding can be defined thermodynamically. It is also particularly useful when binding is weak. The preferential binding coefficient is a measure of the excess number of additive molecules in the domain of the protein per protein molecule (FIG. 4). The connection between the thermodynamic definition (equation 11) and the intuitive notion of binding (local excess number of molecules) comes from statistical mechanics; where it can be shown that:

$${Γ}_{XP} = 〈 {n}_{X}^{II} - {n}_{W}^{II} ( \frac{{n}_{X}^{I}}{{n}_{W}^{I}} ) 〉 \qquad ( 12 )$$

In the above equation, n denotes the number of a specific type of molecule (subscript X for the additive and subscript W for water) in a certain domain (superscript I for a bulk volume outside of the vicinity of the protein and superscript II for a volume in the protein vicinity), and angle brackets denote an ensemble average. Kirkwood, J. G.; Goldberg, R. J. J. Chem. Plays. 1950, 18, 54-57; Schellman, J. A. Biopolymers 1978, 1 7, 1305-1322. Note that Γ_{XP} is independent of the choice of the boundary between the domains, as long as the boundary is far enough from the protein.

If the additive concentration is higher in the vicinity of the protein than in the bulk, Γ_{XP} is greater than zero, and lp is lower in the presence of the additive than in its absence. Denaturants such as urea and guanidinium chloride exhibit this type of binding behavior. The reverse is true for sugars, such as trehalose. In trehalose solutions, there is generally a deficiency of trehalose and an excess of water in the vicinity of the protein. For this “preferential hydration” case, Γ_{XP} is less than zero, and μp is higher in the presence of the additive.

Timasheff pioneered the use of high-precision densitometry to measure preferential binding coefficients for protein-cosolvent systems. Lee, J. C.; Timasheff, S. N. J. Biol. Chem. 1981, 256, 7193-7201; Lee; I. C.; Timasheff; S. N. Biochemistry 1974, 13. 257-265; Gekko, K.; Timasheff, S. N. Biochemistry 1981, 20. 4667-4676; Gekko, K.; Timasheff, S. N. Biochemistry 1981, 20, 4677-4686. More recently, differential scanning calorimetry (DSC) and vapor pressure osmometry (VPO) have been used to the same end. Poklar, N.; Petrovcic. N.; Oblak, M.; Vesnaver; G. Protein Sci. 1999, 8, 832-840; Courtenay, E. S.: Capp, M. W.; Anderson; C. F.; Record Jr., 11. T. Biochemistry 2000, 39, 4455-4471. Preferential binding coefficients are rigorous thermodynamic quantities and are related to virial coefficients, activity coefficients, and free energies via standard thermodynamic relations for multi-component solutions. Casassa. E. F.; Eisenberg, H. Adv. Protein Chem. 1964, 19, 287-395.

Experimental studies by the above methods have led to some generalizations about preferential binding coefficients:
1. Γ_{XP} may be positive or negative, indicating that interactions of the protein and additive are favorable or unfavorable, respectively.
2. Γ_{XP} is proportional to additive molality at low concentration of additive (often as high as mx˜1 m and higher). Courtenay, E. S.: Capp, M. W.; Anderson; C. F.; Record Jr., 11. T. Biochemistry 2000, 39, 4455-4471; Greene Jr., R. F.; Pace. C. N. J. Biol. Chem. 1974, 249, 5388-5393; Record Jr., M. T.; Zhang; W.; Anderson; C. F. Adv. Protein Chem. 1998, 51, 281-353.
3. Γ_{XP} is roughly proportional to the protein-solvent interfacial area. Lee, J. C.; Timasheff, S. N. J. Biol. Chem. 1981, 256, 7193-7201.

The second generalization above, together with the fact that many binary mixtures of additive and water (mp→0) are nearly ideal at low concentration of additive, leads to a useful simplification of equation 10:

$$\begin{matrix}Δ {μ}_{P}^{tr} = - {\int }_{0}^{{m}_{X}} {( \frac{\partial RT ln {m}_{X}}{\partial {m}_{X}} )}_{{m}_{P}} ( \frac{{Γ}_{XP}}{{m}_{X}} ) {m}_{X} d {m}_{X} \\ = - RT ( \frac{{Γ}_{XP}}{{m}_{X}} ) {\int }_{0}^{{m}_{X}} d {m}_{X} \\ = - RT {Γ}_{XP}\end{matrix} \qquad ( 13 ) , ( 14 ) , ( 15 )$$

Equation 15 provides a simple and convenient link between preferential binding coefficients and free energies. This relation leads to the useful rule that when Γ_{XP} is proportional to mx, for each additive molecule that preferentially interacts with the protein, the protein's free energy is reduced by approximately 0.6 kcal/mol at 25° C. The simplicity of this relation is a natural result of the close relationship between Γ_{XP} and a second virial coefficient.

To be able to predict preferential binding coefficients and understand their origins, the above thermodynamic framework and general observations must be augmented by a mechanistic model. Several such models have been presented in the literature, including models based on the binding polynomial or statistical mechanical partition function, solvent-additive exchange at defined sites, additive partitioning between the local and bulk domains, and group contribution methods for estimating transfer free energies.

The most general model of additive binding hitherto presented comes from considering an equilibrium of all possible protein-additive complexes, from which it can be shown that:

$${Δμ}_{P}^{tr} = - RT ln ( 1 + {\sum }_{i} {\sum }_{j} {K}_{ij} {m}_{W}^{i} {m}_{X}^{j} ) \qquad ( 16 )$$

where Kᵢⱼ is the equilibrium constant for a reaction of a protein molecule, i molecules of water, and j molecules of additive into a complex. Wyman, J.; Gill; S. J. Binding and Linkage: Functional Chemistry of Biological Macromolecules: University Science Books: 1990. While this model is completely general, its utility is limited because it is not possible to determine experimentally the many Kᵢⱼ parameters present in equation 16.

Schellman's site exchange model, provides a way to simplify this general expression to a form containing a single parameter. Schellman, J. A. Biopolymers 1978, 17, 1305-1322. This model treats binding as a family of protein-solvent exchange reactions such as:

P·Wᵢ+X→P·X+iW (17)

where P is the protein, W is water, X is cosolvent; and i is the exchange stoichiometry. The simplification requires the assumptions that 1:1 exchange reactions (i=1) occur on a fixed number of identical, independent sites and that the sites are far from saturation with additive (i.e. the apparent dissociation equilibrium constant for each site is well above the additive concentration). The number of sites, n, is approximated by the number of water molecules present in a monolayer around the protein. These simplifications reduce equation 16 to:

Δμ_{P}^{tr}=−nRT [symbol] K [symbol] mₓ (18)

where [symbol] K [symbol] is the average equilibrium constant of binding at a single site. The single parameter [symbol] K [symbol] can then be determined from an experimental measurement of Γ_{XP}. When equation 15 holds, the relation between [symbol] K [symbol] and Γ_{XP} is simply:

[symbol]

K [symbol] =Γ_{XP}/nmₓ (19)

Values of [symbol] K [symbol] for different proteins in this linear regime are roughly equal. Schellman, J. A. Biophys. Chem. 2002, 96. 91-101. [symbol] K [symbol] cannot, however, be determined without knowledge of Γ_{XP} or other free energy data on the particular additive system of interest. In fact, one can say that [symbol] K [symbol] is defined by Γ_{XP}.

Another model that recasts preferential binding coefficient data in terms of a single model parameter is the local-bulk domain model developed by Courtenay et al. Courtenay, E. S.: Capp, M. W.; Anderson; C. F.; Record Jr., 11. T. Biochemistry 2000, 39, 4455-4471. The parameter in this model is the partition coefficient K_{P}, relating the number of water molecules and additive molecules in the local and bulk domains via:

$${K}_{P} = \frac{{n}_{X}^{II} / {n}_{W}^{II}}{{n}_{X}^{I} / {n}_{W}^{I}} \qquad ( 20 )$$

Similar to the site exchange model, the convention used in this model is that the local domain consists of a monolayer of water and enough additive to obtain the experimentally observed Γ_{XP}. Note that because the absolute occupancy of water and additive in the local domain cannot be easily determined by experiment, the local-bulk domain model effectively defines nw. Like [symbol] K [symbol] , values of Kₚ can be used to predict Γ_{XP} at other additive concentrations or for other proteins in the same additive, but predictions cannot be made in the absence of Γ_{XP} or free energy data on the same additive system.

Lastly, transfer free energy models, pioneered by Bolen's group, take a different approach. Liu, Y. F.; Bolen, D. W. Biochemistry 1995, 34, 12884-12891. These models conceptually divide whole proteins into groups such as the amino acid side chains and the protein backbone and model the transfer free energy of the whole protein as a sum of the transfer free energy of the groups it comprises, via:

$$Δ {μ}_{P}^{tr} = {\sum }_{i} {α}_{i} Δ {g}_{i}^{tr} \qquad ( 21 )$$

where Δgᵢ is the transfer free energy of the model group and αᵢ is the solvent accessible area of the group in the whole protein, normalized to the solvent accessible area of the model compound. Tanford, C. J. Am. Chem. Soc. 1964, 86, 2050-2059. The overall Δμ^{tr}ₚ can then be predicted for any system of known structure. In the context of the previously described models, the transfer free energy model can be thought of as a linearized binding model where each surface group or amino acid in the protein represents a different type of independent binding site, and the binding constants for those sites are determined by experiments on model compounds, such as free amino acids or cyclic di-amino acid compounds. Predictions made by transfer free energy models have met with mixed success. A linear group contribution model (equation 21) may be too simple to capture all of the important contributions to Δμ^{tr}ₚ. Bolen, D. W. Protein Stabilizaiton by Naturally Occurring Osmolytes. In Protein Structure, Stability, and Folding; Humana Press: 2001.

While the above models have helped in the understanding of the phenomenon of preferential binding, they generally incorporate strong assumptions, and they necessitate the use of experimental data on highly analogous systems in order to determine model parameters and make predictions. Thus, their uses as predictive tools and as tools to gain insight into specific systems are limited.

One aspect of the present invention relates to a predictive, molecular-level approach for the study of preferential binding based on all-atom, statistical mechanical models that use no adjustable parameters. To date, statistical mechanical models of preferential binding have only been developed for interactions of ions with charged cylinders and for interactions of two-dimensional, “hard circles” with a linear interface, both far too simple to be generally applied to protein-additive systems. Anderson; C. F.; Record Jr., M. T. J. Phys. Chem. 1993, 97, 7116-7126; Mills, P.; Anderson, C. F.; Record Jr., M. T. J. Phys. Chem. 1986, 90, 6541-6548; Tang. K. E. S.: Bloomfield, V. A. Biophys. J. 2002, 82. 2876-2991. Other explicit mixed solvent simulations of proteins and amino acids have been performed, but these studies did not compute thermodynamic quantities related to preferential binding. Zou, Q.; Bennion. B. J.; Daggett, V.; Murphy, K. P. J. Am. Chem. Soc. 2002, 124, 1192-1202; Bennion, B. J.; Daggett, V. PNAS 2003, 100, 5142-5147; Tirado-Rives, J.; Orozco, M.; Jorgensen, W. L. Biochemistry 1997, 36, 7313-7329; Alonso, D. O. V.; Daggett, V. J. Mol. Biol. 1995, 247, 501-520; Caflisch. A.; Karplus, XI. Structt. Fold. Des. 1999, 7, 477-488. In the present invention, the number of “bound” molecules are defined in a thermodynamically consistent way and do not a priori incorporate any information about “binding sites.” The use of this approach for the computation of preferential binding coefficients was validated in two systems by comparison with experimental data from the literature. Additionally, the molecular-level detail of the approach provides new insights into the following issues:
1. The changes in solvent and additive concentration as a function of distance from the protein surface.
2. A precise definition of the “local domain” (FIG. 4).
3. The differences in preferential binding or apparent binding equilibrium constant at different locations on the protein-solvent interface.

The success of this method in modeling preferential binding indicates that it captures the important underlying physics of protein-additive-water systems and that the difficulty in quantitative prediction to date can be surmounted by explicitly incorporating the complex protein-solvent and solvent-solvent interactions.

### A Molecular-Level Approach to Computing Preferential Binding

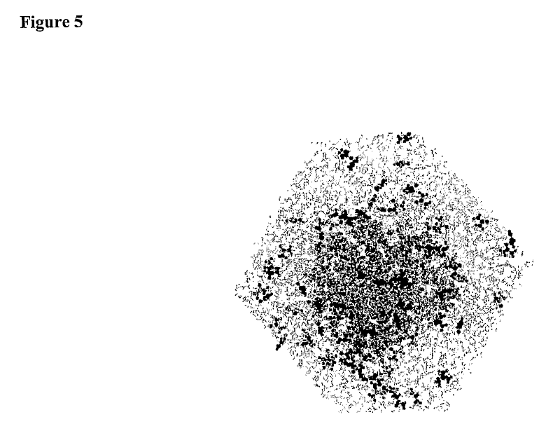

One aspect of the present invention relates to the use of explicit atomic interaction potentials (force fields), such as Lennard-Jones, Coulombic, spring, and torsion interactions, with pre-fit coefficients. Brooks; B. R.; Bruccoleri; R. E.; Olafson, B. D.; States, D. J.; Swaminathan, W.: Karplus, M. J. Comp. Chem. 1983, 4, 187-217; Ha; S. N.; Giammona; A.: Field, M.; Brady, J. W. Carbohydrate Res. 1988, 180, 207-221. Thermodynamic properties, such as preferential binding coefficients, are computed by averaging in the time domain via molecular dynamics (MD). A snapshot from a dynamic simulation of RNase T1 in a urea solution is shown in FIG. 5, which was generated with VMD. Humphrey, W.; Dalke, A.; Schulten, K. J. Molec. Graphics 1996, 14, 33-38. The results of the simulations contain all of the information needed to extract thermodynamic properties, such as Γ_{XP}.

Molecular dynamics uses Newton's second law of motion, that acceleration is the quotient of force and mass, to compute the positions of each atom in the system as a function of time. To do this, an energy model, sometimes called a “force field,” that can be used to compute the net force on any atom in any configuration is employed.

During the MD run, the positions of each atom are recorded at fixed intervals in time. These “snapshots” form an ensemble of configurations which can then be used to compute thermodynamic properties, such as Γ_{XP}.

Importantly, this method of computing Γ_{XP} does not introduce any adjustable parameters to model preferential binding or any other aspect of a system containing a protein and solvent-additive components. All of parameters required by the MD method for energy computations are determined independently of this particular modeling objective, and in fact have been shown to be generally applicable to biological systems. Karplus, M., McCammon, J. A. Nature. Struct. Biol. 2002, 9, 646-652. Thus, the method developed here could be used to estimate Γ_{XP} and Δμ^{tr}ₚ in systems where no experimental data is available. It therefore facilitates the study of preferential binding when direct experimental study is difficult, such as at transition state configurations or at marginally stable states of proteins. Furthermore, it yields detailed, local, molecular-level insight into the system studied.

Another benefit of this approach is that when equation 15 holds (such as for urea and glycerol), the protein transfer free energy (Δμ^{tr}ₚ) can be calculated from a single Γ_{XP} simulation. Traditional free energy calculation methods such as thermodynamic integration require 15-20 trajectories, which is computationally difficult for protein systems of this size. Bash, P. A.; Singh, U. C.: Langridge, R.; Kollman. P. A. Science 87, 236, 564-569; Kollman, P. Chem. Rev. 1993, 93, 2395-2417.

### Preferential Binding Coefficients of Constituent Groups

Because proteins have a range of different functional groups in different orientations on their surfaces, the concentrations of solvents and additives near different patches on the protein's surface may be different. For example, the vicinity of a hydrophobic patch on the protein may have a lower concentration of water and a higher concentration of additive than in the vicinity of a hydrophilic patch. Preferential binding experiments capture only the average effect arising from all of the interactions over the entire protein-solvent interface; however, molecular simulations allow more detailed analyses of the local contributions to preferential binding coefficients.

A protein can be thought of as a set of non-overlapping constituent groups, each of which has its own preferential binding coefficient defined by the composition of the solvent in its immediate vicinity. Tanford, C. J. Am. Chem. Soc. 1964, 86, 2050-2059. Similar to group contribution methods for computing transfer free energies, one possible group definition is that each type of amino acid side chain (up to 20) and the amino acid backbone are distinct groups. To compute a preferential binding coefficient for a constituent group, the solvent molecules in the local domain are assigned only to the nearest group (i), and the “group preferential binding coefficients” (Γ_{XP}, i) can be defined as:

$${Γ}_{XP , i} = 〈 {n}_{X , i}^{II} - {n}_{W , i}^{II} ( \frac{{n}_{X}^{I}}{{n}_{W}^{I}} ) 〉 \qquad ( 22 )$$

where n^{II}_{x,i} and n^{II}_{w,i} are the number of additive and water molecules in the local domain that are nearest to group i. If each additive molecule in the local domain is assigned to a group, the overall preferential binding coefficient is simply the sum of all of the group preferential binding coefficients:

Γ_{XP}=ΣΓ_{XP}, i (23)

The group preferential binding coefficients decompose the effect of each small subset of the protein on the overall preferential binding coefficient. This is analogous to the group contribution models for transfer free energy except that the parameters are extracted from a simulation of an entire protein instead of experiments on model compounds.

### Minimum Simulation Time

Sufficient sampling of position-space configurations in time is required for the accurate calculation of Γ_{XP} via equation 11. Assuming that the average protein solution structure is close to that of the initial (crystal) structure and that water molecules sample position space rapidly because of their high density, the most important time scale to be captured is that of the additives sampling position space. One way to estimate this time is that it must be much larger than the average time between additive-additive contacts.

An estimate of the time between contacts can be obtained as:

$$\begin{matrix} {t}_{contact} \text{?} \frac{1}{12 D} ( \frac{{V}_{solv}}{nx} ) \text{?} & ( 24 ) \\ \text{?} \text{indicates text missing or illegible when filed} & \end{matrix}$$

where D is the additive diffusivity, Vₛₒₗᵥ is the solvent volume, and nx is the number of additive molecules. For the simulations performed here, the solvent is mostly water, so equation 24 can be further simplified to yield:

$${t}_{contact} = \frac{1}{12 D} {( \frac{1}{{N}_{A} {ρ}_{W} {m}_{X}} )}^{\frac{2}{3}} \qquad ( 25 )$$

where N_{A} is Avogadro's number and ρw is the density of water in kg/m³. For a 1 m additive in water system with a additive diffusivity of 2×10⁻⁹ m²/s (a lower bound on the diffusivities of the additives studied here), t_{contact} is about 30 ps. Thus, nanosecond trajectories will be required for good sampling of additive position space. Importantly, this time increases as the additive concentration decreases, implying that there is a minimum concentration that can be studied with any given amount of computational resources.

### Radial Distribution Functions of Water and Additives

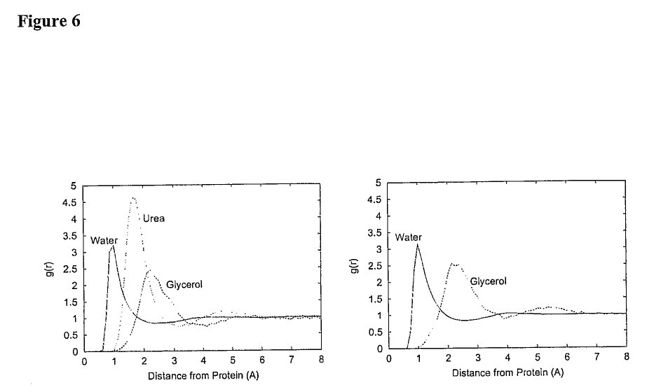

The radial distribution functions of water, urea, and glycerol were computed for all three simulations as described in the Exemplification section and are shown in FIG. 6.

At very short distances, r<0.6 Å for water and r<1.0 Å for glycerol and urea, regions of total solvent and additive exclusion due to very strong van der Waals repulsion can be seen. The size of these “totally excluded” regions is much smaller than one would expect based on the apparent van der Waals radii of the solvent and additive molecules alone (for example, r≈1.5 Å for water and 2.2 Å for urea), indicating that electrostatic attractive forces play an important role in solvation even at these distances. Schellman, J. A. Biophys. J. 2003, 85, 108-125. After the regions of total exclusion, strong first coordination shells of these three molecules can be clearly seen. The peaks of the first coordination shells become more distant from the protein as the size of the molecules they correspond to increases. Significantly smaller second coordination shell peaks are also visible for urea solvating RNase T1 and glycerol solvating RNase A. At distances greater than 6-7 Å from the protein, solvation shells cannot be discerned, and the number densities of water, urea, and glycerol reach their bulk values.

In the simulations of RNase T1 in glycerol and urea solutions, the radial distribution functions for glycerol and urea are quite different. The maximum value of gx(r) for urea is over 4.5, while that for glycerol is about 2.5. The difference in these maximum values, while significant, is not sufficient to say that the number of urea molecules coordinated to the protein (nₓ) is higher than the number of glycerol molecules coordinated, this can only be done by integrating each gx(r) function appropriately via equation 31.

The radial distribution functions for both water and glycerol are similar in the simulations of RNase A and RNase T1 in glycerol solution, despite the fact that the proteins and the pHs of the solutions are different. Given that the proteins are of similar size, this observation is consistent with the fact that the values of Γ_{XP} for the two solutions are close.

### Preferential Binding Coefficients

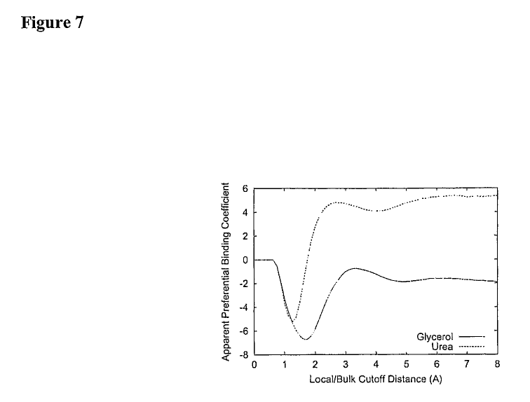

The radial distribution functions in FIG. 6 suggest that r* in the range of 6-8 Å is an appropriate choice of boundary between the local and bulk domains. The error in Γₓₚ introduced by a particular choice of the boundary distance, r*, can be estimated by plotting the apparent preferential binding coefficient (Γₓₚ) versus r* (FIG. 7). Γₓₚ depends very strongly on r* in the first solvation shell (r=0-4 Å) and weakly on r* in the second solvation shell (r=4-6 Å). In the range r=6-8 Å, the dependence of Γₓₚ on r* is small (±0.5), and is less than the statistical error in Γₓₚ (shown in Table 2, explained below). Therefore, a cutoff distance of 6 Å, or about two solvation shells, is sufficiently large to minimize systematic error in Γₓₚ caused by the choice of r*. If only a single solvation shell were considered (r*˜3.5-4 Å), a systematic error in Γₓₚ of approximately 0.5-1 molecules would be introduced as a result of neglect of the second solvation shell.

The preferential binding coefficient, Γₓₚ, was computed via equation 11 using r*=6 Å as the boundary between the local and bulk domains. A confidence interval for this ensemble average was computed as described in the Exemplification section. The binding coefficients and their statistical uncertainties are shown in Table 2.

TABLE 2
Preferential binding coefficients computed from MD
simulations and compared with available experimental
data at similar additive concentrations.

| System | m_{bulk} | Simulation Γ_{XP} | Experimental Γ_{XP} |
|---|---|---|---|
| Urea/Rnase T1 | 1.10 m | 5.2 ± 1.0 | 6.4^{a} |
| Glycerol/Rnase T1 | 1.07 m | −1.6 ± 0.8 | |
| Glycerol/Rnase A | 0.91 m | −0.9 ± 1.0 | −1.7 ± 0.8^{b} |

^{a}Lin, T. Y.; Timasheff, S. N. Biochemistry 94, 33, 12695-12701.
^{b}Gekko, K.; Timasheff, S. N. Biochemistry 1981, 20, 4667-4676.

A wide range of behavior (positive and negative preferential binding coefficients) can be modeled without the use of adjustable parameters. The confidence intervals on Γₓₚ(MD) are an estimate of the statistical error resulting from the use of a finite trajectory. For easier comparison, the experimental values of Γₓₚ reported above were interpolated to m_{bulk} from data sets spanning the molality of interest.

Experimental values from the literature were available for two out of three of these protein-additive systems, and the computed values of Γₓₚ agree quite favorably with these values. The fact that this occurs for both positive and negative values of Γₓₚ without the use of any adjustable parameters is very encouraging. For an additive that obeys equation 15, the confidence intervals of ±1.0 in Γₓₚ represents a confidence limit in the transfer free energy of about 0.6 kcal/mol, which is a typical value for free energies calculated via this type of molecular simulation. Achievement of this level of accuracy despite the fact that structural fluctuations in the native state ensemble of proteins have been observed on much longer time scales than the time scale of the simulations performed here suggests that solvent dynamics are more important than protein structural dynamics in determining Γₓₚ. Duan, Y.; Kollman, P. A. Science 1998, 282, 740-744.

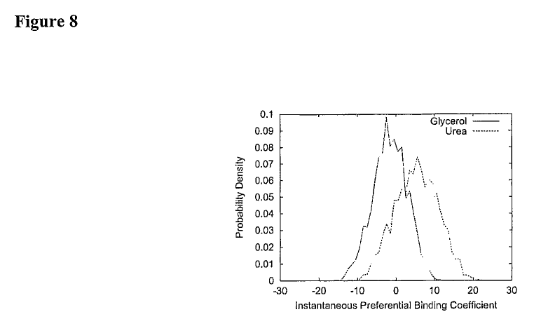

Γₓₚ(t) probability density functions for the simulations of RNase T1 in urea and glycerol solution are shown in FIG. 8. The range of instantaneous values of the preferential binding coefficient, Γₓₚ(t), is quite large relative to the absolute values of Γₓₚ. Γₓₚ(t) values in excess of Γₓₚ±15 are observed. The breadths of these distributions are related to the size of the interface between the local and bulk domains and indicate the importance of sampling a large number of solvent configurations to obtain the macroscopic, averaged Γₓₚ (equation 27).

### The Relation Between Solvent Accessible Area and the Number of Molecules in the Local Domain

The solvent accessible areas of whole proteins (SAA) and constituent groups (SAAᵢ) in crystal structures have been used extensively in analyzing proteins. SAA and SAAᵢ are essentially simple ways of measuring water coordination numbers. In models developed to date, SAA or SAAᵢ, has been used to estimate n_{w} or n_{w,i} by assuming that the local domain is a monolayer of water and each water molecule occupies approximately 10 Å² of the solvent accessible area. Since the present invention introduces a new notion of the local domain, it is worthwhile to see what relationships exist between SAAᵢ and the coordination numbers n_{w,i} and n_{x,i} that utilize this definition.

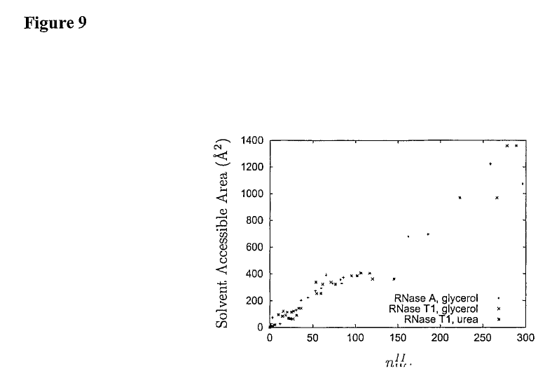

A scatter plot of the solvent accessible area of a set of constituent groups (amino acid side chains and the protein backbone) versus the number of water molecules in the local domain for three different simulations is shown in FIG. 9. Solvent accessible area was calculated analytically in CHARMM (based on Richmond's method) using a 1.4 Å probe. Richmond, T. J. J. Mol. Biol. 1984, 178, 63-89. There is a strong, linear correlation of these variables with slope 4.2 Å²/molecule and correlation coefficient 0.96. Similarly strong correlations are seen for SAAᵢ with n_{x,i} in individual simulations. A summary of proportionality constants and correlation coefficients for these relationships is shown in Table 3. If the time average SAAᵢ from each dynamics simulation is used instead of the crystal structure SAAᵢ values, the correlation coefficients increase slightly. Because the time average solvent accessible areas are higher than those in the crystal structure, the proportionality constants shown in Table 3 also increase.

TABLE 3
Relationships between solvent accessible area in each protein
crystal structure and number of solvent molecules in the local
domain for different protein-additive systems. r²
symbolizes the correlation coefficient.

| | | Avg. Protein | |
|---|---|---|---|
| | | SAA/nᵢ^{II} | |
| Species (i) | Protein | (Å²/molecule) | r² |
| Water | RNase A/T1 | 4.2 | 0.96 |
| 0.91 m Glycerol | RNase A | 290 | 0.96 |
| 1.07 m Glycerol | RNase T1 | 230 | 0.93 |
| 1.10 m Glycerol | RNase T1 | 170 | 0.98 |

### Constituent Group Preferential Binding Coefficients

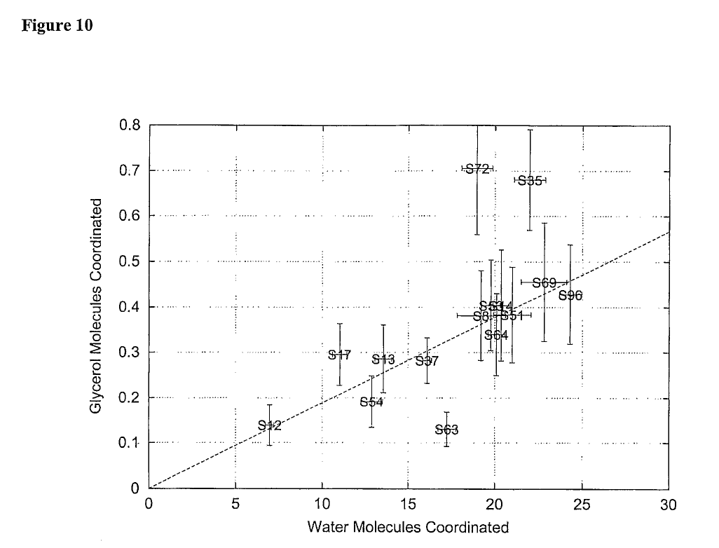

The constituent group preferential binding coefficients were calculated for each simulation as described in the Exemplification section and are shown in FIGS. 10-13 as the number of water and additive molecules coordinated to each constituent group. In each figure, a line at the bulk solution composition is also plotted, enabling a quick determination of the composition of the solvent in the vicinity of a constituent group compared to the bulk solvent. The statistical uncertainties in the values of n^{II}_{w,i} and n^{II}_{x,i} (and consequently Γ_{xp,i}) are high. Because of these uncertainties, we will not report specific values of the group preferential binding coefficients, but rather classify them into broad categories based on their statistical likelihood of being either positive, negative, or zero/indeterminate.

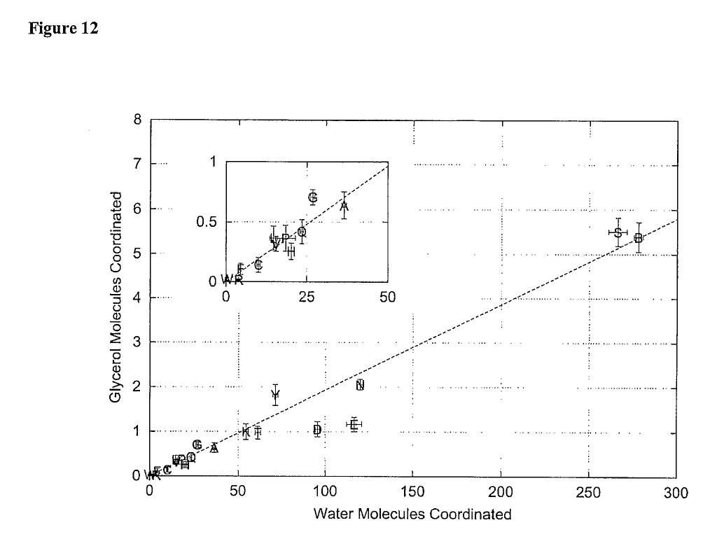

The average number of water and glycerol molecules coordinated to each of the 15 serine residues in RNase T1 are shown in FIG. 10. A wide range of binding behavior can be seen among the serine residues, all of which have a good degree of solvent exposure. Ser 17, 35, and 72 fall above the bulk concentration line and have positive preferential binding coefficients, Ser 63 falls below the line and has a negative preferential binding coefficient, and the preferential binding coefficients of the remaining 11 serine residues are not statistically different from zero. The wide range of local concentrations in the vicinities of these serine residues indicates that developing a group contribution method to estimate Γₓₚ or Δμ^{tr}ₚ based on primary sequence information and solvent accessibility (n^{II}_{w,i}) alone may be difficult. In addition to the type of amino acids present at the protein-solvent interface, other effects such as specific combinations of residues and secondary or tertiary structure must be important in determining water and additive binding behavior. These factors probably contribute to the range of local concentrations seen in FIG. 10. For example, Ser35 and Ser72 are proximal to each other and several Gly and Tyr side chains (Gly 34, 70, 71, and Tyr 68), which tend to have positive preferential binding coefficients in glycerol (FIG. 12). This may be the reason that the group preferential binding coefficients for these residues are higher than those of the other serine residues.

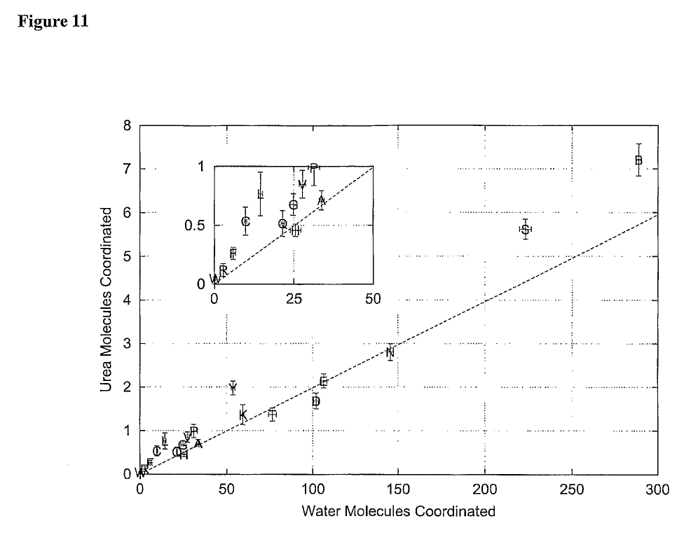

The preferential binding behavior of urea and glycerol, with each type of amino acid in RNase T1 and the protein backbone are shown in FIGS. 11 and 12. In urea solution, the protein backbone and Ser as well as the hydrophobic amino acid side chains of Cys, Gly, Len, Phe, Pro, Tyr, and Val all preferentially bind urea, while the hydrophilic Asp preferentially binds water. In glycerol solution, only Tyr and Gly preferentially bind glycerol, and Asp and Glu preferentially bind water. Qualitatively, the binding behavior of the amino acid side chains of RNase T1 follow a hydrophobic series, with the hydrophobic side chains tending to bind more additive and the hydrophilic ones tending to bind more water.

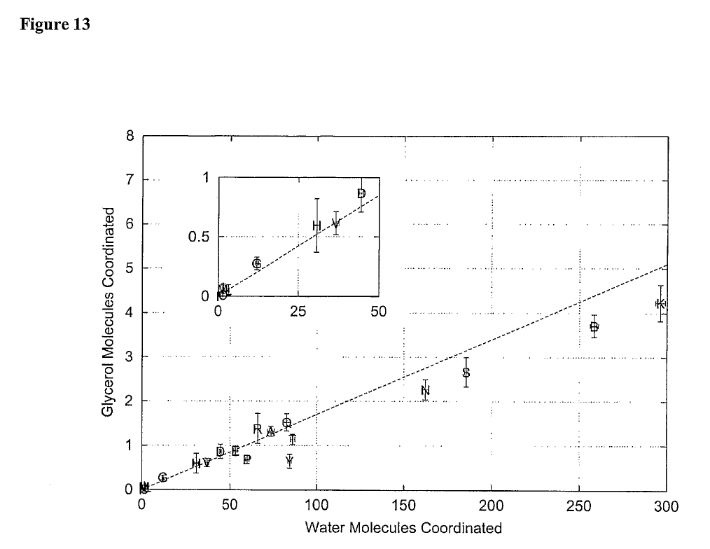

The binding behavior of glycerol and water with the amino acid side chains and backbone in RNase A, shown in FIG. 13, is significantly different than the binding behavior of these solvent components with the same constituent groups in RNase T1. (Note that the protonation states of Asp, Glu, and His are different in the two simulations.) The amino acid backbone, which occupies a large fraction of the protein-solvent interface as indicated by its high value of n^{II}_{w,i}, has a binding coefficient near zero in RNase T1 and a significant negative binding coefficient in RNase A. More strikingly, Tyr in RNase T1 preferentially binds glycerol whereas Tyr in RNase A preferentially binds water. This is likely because the six Tyr residues in RNase A are at or near the solvent interface (a more hydrophilic region) whereas the nine in RNase T1 are mostly buried (a more hydrophobic region). This difference in solvent exposure is evident from the crystal structures of the proteins but also can be discerned by comparing the water coordination numbers for Tyr in the two proteins: n^{II}_{w,i} for Tyr in RNase A is higher than in RNase T1, even though there are 50% more Tyr residues in RNase T1.

Based on the above observations, some generalizations about the effects that these additives have on protein folding equilibria can be postulated, the validity of which must be confirmed via future studies. In urea solution, most of the constituent groups in RNase Ti either preferentially bind urea or are indifferent to urea and water. Asp, which is found on the surface of RNase T1, is the only constituent group that is significantly below the bulk concentration line in FIG. 11 and therefore preferentially binds water over urea. Since the amino acids that compose the core of RNase T1 and are exposed upon unfolding preferentially bind urea, this pattern suggests that the preferential binding coefficient or urea with unfolded RNase T1 is higher than that with native RNase T1. This is thermodynamically consistent with urea's well-known ability as a denaturant. Inversely, in glycerol solution, almost all of the constituent groups in RNase A and T1 are neutral or preferentially bind water. This is consistent with the fact that glycerol binds less to the unfolded protein than the native state, and therefore is a protein stabilizer. Both of these generalizations are consistent with earlier work on model compounds. Bolen, D. W. Protein Stabilizaiton by Naturally Occurring Osmolytes. In Protein Structure, Stability, and Folding; Humana Press: 2001.

### ArgHCl and GuHCl Effect on Globular Protein Association

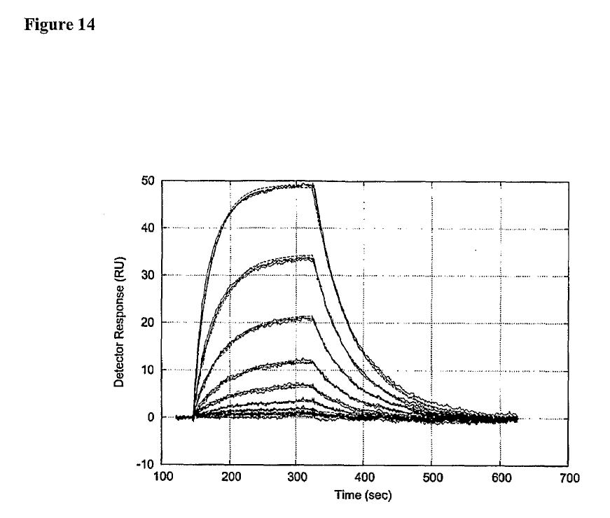

Surface plasmon resonance experiments were conducted to measure the effect of added ArgHCl and GuHCl on the kinetics of globular protein association and dissociation versus an equimolar salt control (NaCl). A typical experimental data set for a binding interaction at one buffer condition is shown in FIG. 14. The data set shown in the figure is a composition of 8 different concentration runs plus replicates, for a total of 16 runs. At t=140 sec, the flow cell with immobilized anti-insulin was exposed to a constant concentration of insulin in the range of 2 to 188 nM for 3 minutes. During this 3 minutes, the antibody and antigen were free to associate and dissociate. The net reaction is the binding of free antigen in solution, resulting in an increase in detector response proportional to the mass of antigen bound. At t=320 sec, the insulin concentration in the flow cell inlet is returned to zero, and the bound antigen then dissociates from the surface. All 16 runs were simultaneously fit to a binding model by minimizing the squared residuals to yield the association and dissociation rate constants, ka and kd. This process was repeated to yield association, dissociation, and equilibrium constant data for the model systems in various buffers as shown in Table 4.

TABLE 4
Effect of arginine on association and dissociation rate
constants for insulin with a monoclonal antibodies.

| Buffer | | | | | |
|---|---|---|---|---|---|
| Additive^{a} | kₐ (M⁻¹s⁻¹)^{c} | k_{d} (s⁻¹)^{c} | K_{D}(μM) | kₐ/kₐ₀^{b} | k_{d}/k_{d0}^{b} |
| 0.5 M NaCl | 4.4 × 10⁴ | 1.4 × 10⁻² | 0.32 | | |
| 0.5 M ArgHCl | 1.2 × 10⁴ | 2.2 × 10⁻² | 1.8 | 0.27 | 1.6 |
| 0.5 M GuHCl | 4.0 × 10⁴ | 9.4 × 10⁻² | 2.4 | 0.91 | 6.7 |

^{a}The base buffer was Biacore HBS-EP (10 mM HEPES, 0.15 M NaCl, 3 mM EDTA, 0.005% polysorbate 20, pH 7.4).
^{b}ka0 and kd0 are the association and dissociation rate constants in HPS-EP + 0.5 M NaCl.
KD ≡ kd/ka.
^{c}The estimated error in the absolute values of ka and kd is 15%.

Relative to the 0.5M NaCl control, 0.5M GuHCl significantly increases the dissociation rate of insulin and anti-insulin and has an insignificant effect on the association rate. This effect of GuHCl on dissociation rate is consistent with its well-known behavior as a strong denaturant. Small denaturants such as guanidinium chloride and urea bind uniformly to protein surfaces and thermodynamically favor protein states which have the largest solvent-accessible area, such as denatured states (in folding equilibria) and dissociated states (in association equilibria). Since GuHCl does not significantly affect the rate of association of insulin and anti-insulin, it is likely that the association transition state does not have a significantly different solvent-accessible area than the dissociated state.

### Mechanistic Interpretation

In the preceding section, we observed that arginine slowed protein-protein association and accelerated dissociation, while guanidinium accelerated dissociation and had little effect on association (Table 4). Here, it is desirable to relate these observations to a mechanistic model of additive effects on protein association reactions.

The process begins by considering the change in a protein reaction rate due to an additive:

k=k₀e^{(Δμ}^{ₚ}^{^{tr}}^{-Δμ}^{ₚ}^{^{tr,‡}}^{)/RT} (26)

where k is the rate constant in the presence of an additive; k0 is the same rate constant the absence of the additive; Δμ^{tr}ₚ is the transfer free energy of the reactant into the additive solution; Δμ^{tr}ₚ^{‡} is the transfer free energy of the transition state into the additive solution; R is the gas constant; and T is the absolute temperature. The effect of a particular additive enters into the above equation entirely through the difference in the transfer free energies.

When a high concentration of an additive (>0.1 M) is required to have a significant effect on a protein reaction rate or equilibrium constant, such as has been observed in this study for arginine and guanidinium (data at low concentration not shown), the strength of the additive effect can be termed “weak.” If, in addition to being weak, the additive interacts with the protein at a large number of sites distributed uniformly over the protein's surface, or does not act in a site-specific maimer, the transfer free energy due to the additive is proportional to the solvent accessible area of the protein (aP) and an additive-dependent constant (γX) related to the preferential binding coefficient [Lee, J. C. & Timasheff, S. N. (1974) Biochemistry 13, 257-265; Gelko, K. & T1 masheff, S. N. (1981) Biochemistry 20, 4667-4676; Arakawa, T. & Timasheff, S. N. (1985) Biophys. J 47, 411-414; T1 masheff, S. N. (2002) PNAS 99, 9721-9726; Davis-Searles, P. R., Saunders, A. J., Erie, D. A., Winzor, D. J., & Pielak, G. J. (2001) Annu Rev Biophys Biomol Struct 30, 271-306; Baynes, B. M. & Trout, B. L. (2004) Rational design of solution additives for the preventing of protein aggregation, Biophys. J. 87, 1631-1639]:

Δμₚ^{tr}=−RT_{γXαPcX} (27)

where cX is the concentration of additive. Analogous expressions are frequently used to model the effects of additives such as guanidinium, trehalose, and sorbitol.

The experimental observation that guanidinium does not significantly alter the rate of association of insulin and anti-insulin suggests that the surface area of the pair of molecules accessible to guanidinium does not change significantly from the dissociated state to the association transition state. If this is the case, and if arginine interacts with proteins in the same way that guanidinium does, it should not be possible for arginine, acting in a weak and nonspecific manner, to exert any effect either, yet we observe 0.5M arginine induces approximately a factor of 3 depression in the association rate (Table 4). This suggests that arginine acts via a mechanism distinct from that of guanidinium.

As discussed previously, if an additive is much larger than water but does not significantly affect the free energy of dissociated protein molecules, the additive will increase the activation free energy for the molecules to associate. This steric effect, which is referred to as “the gap effect,” slows protein association and may either speed or slow dissociation.

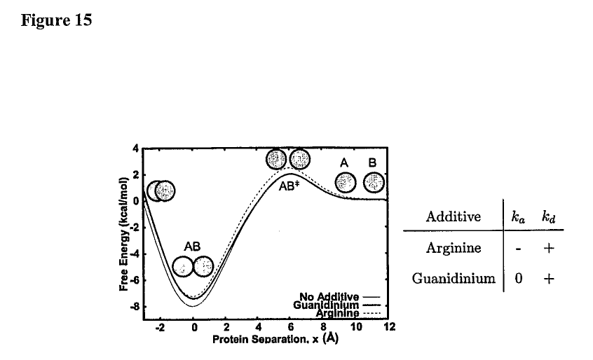

This model can be used to calculate the effects of guanidinium and arginine as described in Example 7. The results of such a calculation are shown in FIG. 15. In the presence of arginine, the model predicts that the free energy of the transition state will increase relative to the dissociated state. This causes the association rate constant to decrease. Inversely, the free energy of the associated state increases relative to the free energy of the transition state, causing the dissociation rate constant to increase. In stark contrast to the arginine effect, the presence of guanidinium has little effect on the transition state free energy relative to the dissociated state, hence guanidillium has no effect on the association rate constant. The associated state free energy, however, increases relative to the transition state, causing the dissociation rate constant to increase. All of these effects are qualitatively consistent with the changes in the measured rate constants for insulin and anti-insulin (Table 4).

Using this model and an analogous model in which the proteins are approximated as planar surfaces, the range of association rate effects caused by arginine can be quantitated. Baynes, B. M. & Trout, B. L. Biophys. J., 2004 87, 1631-1639. The spherical and planar models give a range of 0.8 -2.8 kcal/mol/M for the maximum increase in the free energy barrier to association. For 0.5M arginine solution, this is 0.4 -1.4 kcal/mol, or a rate effect of kₐ/kₐo=e^{-ΔΔμ}^{^{tr}}^{/RT}=0.51 to 0.10. This range covers the experimentally observed value for the association rate depression of insulin and anti-insulin at 0.5M ArgHCl (kₐ/kₐo=0.27, Table 4).

### Effect on Refolding of Carbonic Anhydrase

To assess whether the effects of arginine and guanidinium on globular protein association reactions carry over to a more complex aggregation situation, we examined the effects of eqimolar amounts of NaCl, GuHCl, and ArgHCl on the refolding of carbonic anhydrase II (CA). CA is a natural enzyme that is known to aggregate during refolding.

In previous studies in our laboratory and others, carbonic anhydrase II was found to refold from a denatured state by sequential formation of a molten intermediate state (M), a near-native conformation that has no biological activity (I), and finally the native state (N). Cleland, J. L., Hedgepeth, C., & Wang, D. I. C. 1992 J. Biol. Chem. 267, 13327-13334; Wetlaufer, D. B. & Xie, Y. 1995 Protein Sci. 4, 1535-1543; Semisotnov, G., Rodionova, N. A., Kutyshenko, V. P., Ebert, B., Blanck, J., & Ptitsyn, O. B. 1987 FEBS Letters 224, 9-13; Semisotnov, G. V., Uversky, V. N., Sokolovsky, I. V., Gutin, A. M., Razgulyaev, O. I., & Rodionova, N. A. 1990 J. Mol. Biol. 213, 561-568; Dolgikh, D. A., Kolomiets, A. P., Bolotina, I. A., & Ptitsyn, O. B. 1984 FEBS Letters 165, 88-92; Cleland, J. L. (1991) Mechanisms of protein Aggregation and Refolding, PhD thesis, MIT; Cleland, J. L. & Wang, D. I. C. 1992 Biotechnol. Prog. 6, 97-103; Cleland, J. L. & Wang, D. I. C. 1990 Biochemistry 29, 11072-11078.

U→M→I→N (28)

Cleland showed that the molten intermediate (M) can aggregate to form dimers and higher mers. Cleland, J. L. (1991) Mechanisms of Protein Aggregation and Refolding, PhD thesis, MIT.

M→A₂→(etc.) (29)

In 1.0M GuHCl and at low concentration of carbonic anhydrase (less than 30 μM), the formation of small mers was reversible, leading to yields of native protein approaching 100%. At lower GuHCl concentrations, formation of large aggregates occurred, resulting in significant losses of CA. At long times (hours to days), the only aggregate species observed were small multimers and very large, micron-sized aggregates. These observations lead to the following two predictions about the performance of ArgHCl and GuHCl as solution additives:

1. The reversibility of small multimer formation implies that early association reactions are at least partially equilibrium-controlled. Then, since ArgHCl and GuHCl shift equilibrium toward the smaller mers (Table 4), they both should promote formation of the native protein during refolding. This was probed experimentally by measuring the native protein concentration as a function of refolding buffer conditions.

2. The absence of intermediate-sized aggregates at long times implies that CA aggregation proceeds via a nucleation-dependent polymerization mechanism where a small multimer is the nucleus. After formation of the nucleus, association is rapid and dissociation is negligible. Since ArgHCl deters association, arginine should decrease the average aggregate size and molecular weight in this regime. Conversely, since guanidinium chloride affects the association equilibrium by increasing the dissociation rate, it will have a negligible effect on this regime of aggregation. This was probed experimentally by measuring the multimer distribution as a function of refolding buffer conditions via size exclusion HPLC, as described below.

### Yield of Native Protein

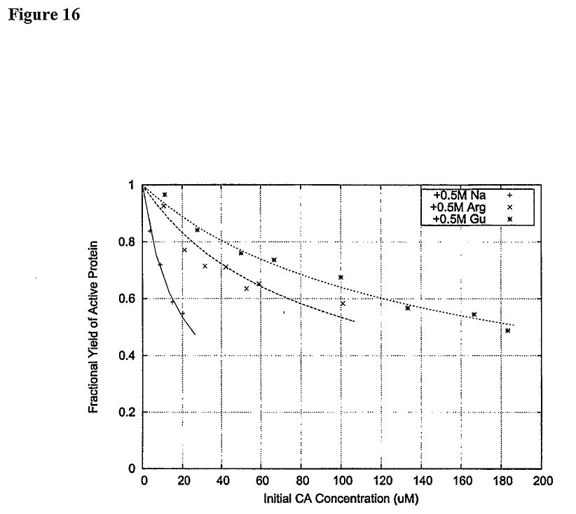

Esterase activity assays were performed as a function of initial unfolded protein concentration and buffer composition to determine how equimolar concentrations of NaCl, ArgHCl, and GuHCl each affected refolding yield (FIG. 16). It was observed that the yield of active protein as a function of buffer additive increased in the following order:

NaCl<<ArgHCl<GuHCl.

If association and aggregation can account for the majority of the loss of native protein, then it should be possible to model the yield of native protein as a function of the initial protein concentration and a parameter characterizing the competition between refolding and aggregation. Hevehan, D. L. & Clark, E. D. B. (1997) Biotechnol. Bioeng. 54, 221-230. Assuming the unfolded protein rapidly collapses to the molten intermediate when introduced into refolding conditions, refolding and aggregation from the molten state can be modeled as being in direct kinetic competition [Semisotnov, G., Rodionova, N. A., Kutyshenko, V. P., Ebert, B., Blanck, J., & Ptitsyn, O. B. 1987 FEBS Letters 224, 9-13; Zettlmeissl, G., Rudolph, R., & Jaenicke, R. 1979 Biochemistry 18, 5567-5571]:

$$N {}^{{k}_{r}} M {}^{{k}_{a g g}} Aggregates \qquad ( 30 )$$

where kr is the refolding rate constant and kagg is the aggregation rate constant.

Since refolding is a unimolecular reaction, it is expected that the refolding reaction is first-order. The kinetic order of the macroscopic aggregation reaction, however, cannot be predicted in advance. In an earlier study of carbonic anhydrase refolding via dynamic light scattering, Cleland and Wang proposed a 2.6-power relationship between initial protein concentration and monomer depletion rate at short times (30-60 sec). Cleland, J. L. & Wang, D. I. C. 1990 Biochemistry 29, 11072-11078. Thus, we expect a reaction order of between 2 and 3 to be applicable in this case. Model cases for aggregation reaction orders of 2 and 3 were fit to the data and revealed that a macroscopic second-order aggregation reaction gave a much better fit for all three buffer conditions. The activity data with added 0.5M GuHCl and 0.5M ArgHCl are suggestive of slightly higher inactivation order than the added 0.5M NaCl case, but because of the uncertainty (±5%) in the esterase activity data, it is not possible to determine the reaction order to better than about ±0.5 by direct fitting.

For a second order aggregation reaction, the yield of native protein is:

$$Yield = \frac{{k}_{r}}{{{k}_{a g g} [ U ]}_{0}} ln ( 1 + \frac{{{k}_{a g g} [ U ]}_{0}}{{k}_{r}} ) \qquad ( 31 )$$

where [U]0 is the initial concentration of unfolded protein. Since the constants kr and kagg appear only as a quotient, they can be condensed to a single “refolding selectivity parameter,” a≡kr/kagg, having units of concentration and resulting in a working equation:

$$Yield = \frac{α}{{[ U ]}_{0}} ln ( 1 + \frac{{[ U ]}_{0}}{α} ) \qquad ( 32 )$$

Each of the data sets in FIG. 16 were fit to the above model equation, yielding the values of a shown in FIG. 15. The functional forms of the model at these values of a are shown in FIG. 16. The parameter a is a direct measure of the performance of a refolding additive. It is equal to the concentration of unfolded protein at which the refolding yield will be ln(2), or about 70%.

The relative refolding selectivity values (a/a0) for ArgHCl and GuHCl indicate that both these additives promote refolding. This supports the notion that formation of irreversible aggregates is at least partially equilibrium-controlled. The refolding selectivity values are also qualitatively consistent equilibrium shifts effects seen in globular protein association (Table 5).

TABLE 5
Refolding selectivity parameters (α) and parameters
relative to 0.5M NaCl (α/α0) are shown for refolding
of carbonic anhydrase with three different buffer additives.
The base buffer composition was 0.5 M GuHCl.

| Additive | α (μM) | α/α₀ |
|---|---|---|
| 0.5 M NaCl | 9.3 | 1 |
| 0.5 M ArgHCl | 47 | 5.0 |
| 0.5 M GuHCl | 77 | 8.2 |

### Multimer Distribution

Size exclusion HPLC experiments were performed to analyze the distribution of multimers formed during refolding. CA was refolded with three different additives, 0.5M NaCl, 0.5M GuHCl, and 0.5M ArgHCl, relative to a base refolding buffers of 0.5M GuHCl, as done in the esterase activity assays above. The 0.5M NaCl refolding experiment was performed at 4-fold lower concentration (5 μM) because visible aggregates were formed within seconds at concentrations comparable to the other two experiments (20 μM). Other than this protein concentration difference, these experiments allow direct comparison of how an additional 0.5M of the three different cations affect refolding.

After initiating refolding by diluting denatured CA with an appropriate buffer, refolding was allowed to proceed for at least two hours before performing HPLC. The samples were not filtered prior to introduction into the HPLC column. The molecular weight distributions observed are shown in Table 6.

In 0.5M NaCl, the refolded carbonic anhydrase is partitioned entirely between monomers and large aggregates, with no significant mass observed in intermediate species. With 0.5M ArgHCl or GuHCl added, the yield of monomeric protein is significantly increased, consistent with the observation of a larger native protein yield in the previous section.

TABLE 6
HPLC analysis of multimers formed during refolding of carbonic
anhydrase in different buffers, expressed as a percentage of
the total carbonic anhydrase.

| | | | | | |
|---|---|---|---|---|---|
| Time | | | | | |
| (min)^{a} | M^{b} | A₂ | A₃₋₅ | A₆₋₁₅ | Large^{c} |

(a) Additive 0.5 M NaCl, [U]₀ = 5 μM

| | | | | | |
|---|---|---|---|---|---|
| 2 | 56 | 0 | 0 | 0 | 44% |
| 20 | 56 | 0 | 0 | 0 | 44% |
| 38 | 56 | 0 | 0 | 0 | 44% |

(b) Additive: 0.5 M ArgHCl, [U]₀ = 20 μM

| | | | | | |
|---|---|---|---|---|---|
| 2 | 22 | 30 | 25 | 21 | 2% |
| 20 | 54 | 7 | 14 | 26 | −1% |
| 38 | 62 | 4 | 11 | 24 | −1% |
| 1500 | 80 | 0 | 0 | 19 | 1% |

(c) Additive: 0.5 M GuHCl, [U]₀ = 20 μM

| | | | | | |
|---|---|---|---|---|---|
| 2 | 42 | 39 | 8 | 0 | 11% |
| 20 | 82 | 3 | 6 | 0 | 9% |
| 38 | 85 | 1 | 5 | 0 | 9% |
| 1500 | 89 | 0 | 2 | 0 | 9% |

^{a}The time reported is the time between injection onto the HPLC column and dilution of the denatured carbonic anhydrase into the refolding buffer. The base refolding buffer contained 0.5M GuHCl.
^{b}M indicates monomer, and Aᵢ₋ⱼ indicates multimers of mer number i through j.
^{c}The amount of “Large” multimers which do not pass through the column is inferred from the difference between the amount of protein injected onto the column and the total chromatogram area. The reproducibility of any peak area determination from experiment to experiment is ±1%.

In all three refolding buffers, significant amounts of large aggregates form which do not dissociate into monomeric protein. With longer refolding times, the average aggregate molecular weight and hydrodynamic radii continue to increase and monomer is slowly depleted (data not shown). This implies that the native protein and large aggregate states are separated by a large free energy barrier.

The average aggregate molecular weight (ignoring the monomer) is lowest in O.5M ArgHCl, despite the fact that 0.5M GuHCl results in the highest yield of native protein. Since intermediate aggregates (A₆₋₁₅) are not observed in 0.5M NaCl or 0.5M GuHCl, but larger aggregates are observed, association must be rapid through the intermediate size range in these buffers. Because dissociation is negligible in such a regime, additives like guanidinium that affect association equilibria through the dissociation rate cannot deter association here. In contrast, arginine, which slows association reactions, can deter formation of higher mers and ultimately leads to a lower average aggregate molecular weight than GuHCl or NaCl.

This type of difference may have important consequences when comparing the performance of different buffer additives via simple surrogate assays. As seen in the differences in yield and aggregate molecular weight distribution between the refolding buffer additives ArgHCl and GuHCl (FIG. 16), a decrease in the average aggregate molecular weight may not be indicative of increased refolding yield. Thus, simple aggregation assays such as turbidity and dynamic light scattering, which roughly measure the amount of large particles in solution, will also not correlate with yield when comparing additives that affect association with those that affect dissociation.

The presence of arginine in solution was shown to slow protein-protein association reactions in two model systems: the association of insulin with a monoclonal antibody, and the association of folding intermediates and aggregates of carbonic anhydrase II (CA). In CA refolding, arginine promoted formation of the native protein and decreased the average molecular weight of CA aggregates.

The denaturant guanidinium chloride (GuHCl), which is also used to dissolve aggregates and deter aggregation in certain situations, exhibited significantly different kinetic behavior than arginine-HCl. GuHCl significantly increased the dissociation rate constant of insulin and anti-insulin and had a negligible effect on their association rate. GuHCl also significantly increased CA refolding yield, but because of the difference in kinetic effects, GuHCl had a smaller effect on reducing the average molecular weight of CA aggregates than ArgHCl.

The magnitudes of the observed effects were quantitatively consistent with gap effect theory. Baynes, B. M. & Trout, B. L. Biophys. J. 2004 87,1631-1639. Arginine and derivatives thereof can be modeled as a “neutral crowder,” an additive that is larger than water but has a negligible effect on the free energy of isolated protein molecules.

The beneficial effect of arginine and derivatives thereof on protein refolding arises because it slows protein association reactions. Thus, in addition to being a useful refolding buffer additive, arginine and derivatives thereof should prevent aggregation in any application where aggregation exhibits second or higher-order kinetics.

### Exemplification

The invention now being generally described, it will be more readily understood by reference to the following examples, which are included merely for purposes of illustration of certain aspects and embodiments of the present invention, and are not intended to limit the invention.

Proteins and Reagents—Human insulin (I8530), bovine carbonic anhydrase II (CA) (C2522), hen egg white lysozyme (L765 1), and bovine serum albumin (B4287) were obtained from Sigma-Aldrich (St. Louis, Mo.). Monoclonal anti-insulin (10-130 clone M322214) was obtained from Fitzgerald Industries (Concord, Mass.). Consumable reagents for Biacore experiments (NHS, EDC, ethanolamine, glycine, and HBS-EP buffer) were obtained from Biacore AB (Switzerland). Guanidinium chloride, arginine hydrochloride, and sodium chloride were attained from Sigma-Aldrich in the highest available grade.

Concentration of carbonic anhydrase in solution was determined by absorbance at 280 nm using an extinction coefficient of 54000 M⁻¹ cm⁻¹. Pocker, Y. & Stone, J. T. (1967) Biochemistry 6, 668-678.

Globular Protein Association Kinetics—Protein association and dissociation rate constants, ka and kd, were measured for globular proteins via surface plasmon resonance on a Biacore 3000 instrument. Monoclonal anti-insulin was immobilized on a Biacore CM5 sensor chip via amine coupling. The amount of immobilized antibody was selected to give a detector response in the range of 50-100 RU when antigen was present. A reference surface was created by activating and deactivating the surface without coupling an antibody to it.

Different concentrations of insulin in the nanomolar range (1-200 μM) were prepared by dilution and injected serially into the antibody-containing and reference flow cells. Such low concentrations were used to ensure that multimerization of insulin did not affect the results. Pocker, Y. & Biswas, Subhasis, B. (1981) Biochemistry 20, 4354-4361. The dissociation rate was sufficiently fast in buffer that a regeneration buffer was not required. Kinetic constants were extracted by simultaneous fitting of ka and kd to each set of sensorgrams using a 1:1 kinetic model in the BIAevaluation 3.0 software package.

Size Exclusion HPLC—Size exclusion HPLC (SE-HPLC) experiments were performed on a Beckman System Gold HPLC instrument equipped with a Tosohaas G3000SWXL size exclusion column and a UV detector. 30 μl samples were introduced to the column by a constant flow of 1 ml/min mobile phase. Each sample ran for 15 minutes, with carbonic anhydrase eluting between 6 and 10 minutes, depending on its molecular weight and buffer. Protein was observed at the exit of the column via absorbance at 280 nm. For samples that did not contain large submicron or micron-sized aggregates (which do not pass through the column), the total chromatogram areas at 280 nm were consistent to within 2-3% during the entire refolding process, indicating that the extinction coefficients of different sized aggregates did not vary significantly on a mass basis. A mixture of lysozyme, carbonic anhydrase, and bovine serum albumin (monomer and dimer) was used as a standard to calibrate molecular weight to retention time. Using this calibration curve and the breakthrough time of the column, the largest multimer that could pass through the column was a 15-mer. When significant mass was missing from a chromatogram, large multimers were quantitated by difference. The presence of large multimers was confirmed via turbidity or dynamic light scattering for each buffer. The instrument was cleaned with 30 μl injections of 4M GuHCl, a denaturing concentration found to dissociate and elute precipitates and large soluble carbonic anhydrase multimers.

### EXAMPLE 1

Molecular Simulations—Molecular dynamics was used to sample the phase space of proteins solvated by water and an additive. Version 28 of the CHARMM molecular dynamics package was used for all simulations. Brooks; B. R.; Bruccoleri; R. E.; Olafson, B. D.; States, D. J.; Swaminathan, W.: Karplus, M. J. Comp. Chem. 1983, 4, 187-217. The CHARMM force-field was used for the protein, and the TIP3P model [32] was used for water. Jorgensen, W. L.; Chandrasekhar. J.; Madura, J. D.; Impey, R. W.; Klein, M. L. J. Chem. Phys. 1983, 79, 926-935. A force-field was constructed for glycerol using the standard CHARA-II\-1 geometries and partial charges for the atoms in a —CHOH— unit. Brooks; B. R.; Bruccoleri; R. E.; Olafson, B. D.; States, D. J.; Swaminathan, W.: Karplus, M. J. Comp. Chem. 1983, 4, 187-217; Ha; S. N.; Giammona; A.: Field, M.; Brady, J. W. Carbohydrate Res. 1988, 180, 207-221. Urea was assumed to be planar with bond lengths equal to the CHARMM standards and partial charges recomputed as done previously [33] but using the CHARMM van der Waals mixing rules in the objective function. Duffy. E. M.; Severance. D. L., Jorgensen, W. L. Israel J. Chem. 1993, 33, 323-330.

The structures of RNase A (PDB code: 1fs3) and RNase T1 (PDB code: lygw) were obtained from the Protein Data Bank. Berman, H. M.; Westbrook, J.; Feng, Z.; Gilliand; G.; Bhat; T. N.; Weissig, H.; Shindyalov. I. N.; Bourne, P. E. Nucleic Acids Res. 2000, 28, 235-242. In total; three simulations were performed: RNase A in 1 m glycerol (pH 3), RNase T1 in 1 m glycerol (pH 7), and RNase T1 in 1 m urea (pH 7). Details of each simulation are shown in Table 7. Each protein was solvated in a truncated octahedral box extending a minimum of 9A from the protein. The pH of each simulation was fixed by setting the protonation states of each ionizable side chain to the dominant form expected for each amino acid at the pH of interest. Arginine, cysteine, lysine, and tyrosine were protonated in all of the simulations. Aspartate, glutamate, and histidine were assumed to have pKa values of 3.4, 4.1, and 6.6, respectively; and were therefore protonated in the simulation at pH 3 and deprotonated at pH 7. Forsyth, W. R.; Antosiewicz. J. hl.; Robertson, A. D. Proteins 2002, 48, 388-403; Edgecomb, S. P.; Murphy, K. P. Proteins 2002, 49, 1-6. Initial placement of water and additive molecules were random. Protein counterions were placed using SOLVATE 1.0. The system was first energy minimized at 0 K, next heated to 298.15 K, and then equilibrated for 1 nanosecond in the NTP ensemble at one atmosphere. For the computation of the properties of interest, two nanoseconds of dynamics were then run, during which statistics were computed from snapshots of the trajectory every picosecond.

TABLE 7
Details of four molecular dynamics (AID) simulations performed.

| Additive | Protein | T (° C.) | pH | nₓ | n_{w} | <l> (Å) |
|---|---|---|---|---|---|---|
| Urea | RNase T1 | 25 | 7 | 90 | 4274 | 57.48 |
| Glycerol | RNase T1 | 25 | 7 | 87 | 4582 | 59.24 |
| Glycerol | RNase T1 | 25 | 3 | 90 | 5480 | 62.86 |

nx is the number of additive molecules,
n_{w} is the number of water molecules, and
<l> is the average dimension of the primary unit cell (which varies during the run at constant pressure).

### EXAMPLE 2

Calculation of Preferential Binding Coefficients—The trajectories were then used to define the local and bulk regions and compute Γₓₚ in the following manner. For the purpose of computing Γₓₚ and other thermodynamic and structural parameters, each water and additive molecule was treated as a point at its center of mass. The distance of each of these points to the protein's van der Waals surface was computed, and then ρw(r) and ρx(r), defined as the number densities of these points at a distance r from the protein, were computed. In all cases, the ρ(r) functions exhibited peaks and valleys characteristic of solvation shells in the range 0 <r <6A. At distances in the range of 6-SA and higher, such variations are no longer seen, and the local number density is defined as bulk number density, ρ(∞). Such a region far from the protein containing a spatially uniform concentration of water and additive must be present in the simulation cell in order to define the local and bulk regions and calculate Γₓₚ.

The position of the boundary between the local and bulk domains, a distance of r* away from the surface of the protein, was then determined by choosing the minimum distance at which no significant difference between ρ(r*) and ρ(∞) was apparent for either water or additive. All solvent molecules whose centers of mass fell inside a distance of r* from the protein's van der Waals surface were defined as belonging to the local domain (II), and all other solvent molecules were defined as belonging to the bulk domain (I). With these definitions of the domains, the instantaneous preferential binding coefficient, Γₓₚ(t), was computed as

$${Γ}_{XP} ( t ) ≡ {n}_{X}^{II} - {n}_{X}^{I} ( \frac{{n}_{W}^{II}}{{n}_{W}^{I}} ) \qquad ( 33 )$$

for each time point in each trajectory. The preferential binding coefficient, Γₓₚ, was then computed for each trajectory as the time average of these instantaneous values:

$${Γ}_{XP} = \frac{1}{t} {\int }_{0}^{t} {Γ}_{XP} ( {t}^{′} ) d {t}^{′} \qquad ( 34 )$$

The radial distribution functions gx(r) and gw(r) are defined as:

gᵢ(r)≡ρᵢ (r)/ρᵢ(∞) (35)

where i represents water (W) or an additive (X) species. These functions provide another route to compute Γₓₚ:

$$\begin{matrix}{Γ}_{XP} = 〈 {n}_{X}^{II} 〉 - 〈 ( \frac{{n}_{X}^{I}}{{n}_{X}^{I}} ) {n}_{W}^{II} 〉 , & ( 36 ) \\ = {ρ}_{X} ( ∞ ) \int {g}_{X} d V - ( \frac{{ρ}_{X} ( ∞ )}{{ρ}_{W} ( ∞ )} ) {ρ}_{W} ( ∞ ) \int {g}_{W} d V , & ( 37 ) \\ = {ρ}_{X} ( ∞ ) \int ( {g}_{X} - {g}_{W} ) d V & ( 38 )\end{matrix}$$

where each integral is over the local domain or the entire system (since gx−gw=0 in the bulk domain).

The boundary between domains I and II must be placed far enough from the protein to ensure that it is in the bulk, yet at the smallest such distance so that statistical fluctuations in the number of molecules in the domains can be minimized. One can use the values of gx(r) and gw(r) to determine the optimal boundary. Defining Γₓₚ as the apparent preferential binding coefficient resulting from defining the local domain as those molecules whose centers of mass lie inside a distance r* from the protein:

$${Γ}_{XP}^{*} ( {r}^{*} ) = {ρ}_{X}^{∞} {\int }_{0}^{{r}^{*}} ( {g}_{X} - {g}_{W} ) \frac{d V}{d r} d r \qquad ( 39 )$$

The error in Γₓₚ, E_{Γ}, introduced by selecting a particular value of r* is then

$$\begin{matrix}{E}_{Γ} = {Γ}_{XP}^{*} - {Γ}_{XP} , & ( 40 ) \\ = - {ρ}_{X} ( ∞ ) {\int }_{{r}^{*}}^{∞} ( {g}_{X} - {g}_{W} ) \frac{d V}{d r} d r & ( 41 )\end{matrix}$$

When r* is selected properly, the surface defined by r=r* is entirely in the bulk solution, gx(r*)=gw(r*)=1, and E_{Γ}=0. Thus, selecting r* as the minimum distance for which all r≧r* satisfy gx(r)=gw(r)=1 (within the error of the simulation) is optimal.

### EXAMPLE 3

Calculation of Constituent Group Preferential Binding Coefficients—For each simulation, up to 21 constituent group preferential binding coefficients were calculated. The 21 groups were each type of amino acid side chain present in the protein (up to 20) and the protein backbone. The “protein backbone” was defined as the —NH—CH—COO— unit, as well as the two extra protons at the N-terminus and extra oxygen atom at the C-terminus of the protein. The glycine side chain was defined as the proton bound to the alpha carbon that would be replaced by a substituent to form a different L-amino acid.

For the simulation of RNase T1 in glycerol solution, the constituent group preferential binding coefficients for the 15 individual serine residues in the protein were also calculated. For this calculation, solvent and additive molecules that were nearest to an atom in the protein that was not part of a serine side chain were not considered.

Water and additive molecules were associated with a specific constituent group by computing the distance from the center of mass of each solvent molecule to the van der Waals surface of every atom in the protein, selecting the protein atom that was nearest to the solvent molecule, and then determining to what constituent group this nearest protein atom belonged.

### EXAMPLE 4

Estimation of Statistical Error—The statistical error arising from computing averaged properties from a finite trajectory was estimated in the following fashion:
1. The dynamic trajectory of interest was divided into n pieces.
2. The mean of the property of interest was computed in each piece. These means were designated zᵢ where i=1 . . . n.
3. The standard deviation of the zᵢ values was computed.
4. This standard deviation was divided by n and the quotient was designated σₘ, an estimate of the error in the mean determined by time averaging the fall trajectory.
   The number of pieces n into which the trajectory is divided must be small enough to ensure that the means of each piece (the zᵢ) are statistically independent. An autocorrelation analysis (not shown) of several trajectories of Γₓₚ(t) data and the underlying molecular counts (nᵢ and nᵢ) indicates that a window of about 0.2 ns is sufficiently large for this to be true. Therefore, for a 2 ns dynamics trajectory, a value of n=2/0.2=10 was used.

For long trajectories, the statistical error σₘ is roughly proportional to the inverse square root of the trajectory length. This property can be used to estimate the trajectory length required to achieve a given level of statistical accuracy after a small trajectory has been generated and analyzed.

### EXAMPLE 5

Refolding of Carbonic Anhydrase—Refolding of carbonic anhydrase was accomplished by dilution from high concentrations of the denaturant guanidinium chloride (GuHCl) as done previously. Cleland, J. L., Hedgepeth, C., & Wang, D. I. C. (1992) J. Biol. Chem. 267, 13327-13334; Wetlaufer, D. B. & Xie, Y. (1995) Protein Sci. 4, 1535-1543. High concentrations of carbonic anhydrase (>300 μM) were denatured in 6M GuHCl and equilibrated overnight. Refolding was initiated by dilution to 0.5M GuHCl with 50 mM Tris-HCl buffer, pH 7.5. This final GuHCl concentration was selected because it yields a mixture of active, refolded protein and aggregates. The distribution of this mixture was analyzed via esterase activity, size exclusion HPLC, and dynamic light scattering as described above.

### EXAMPLE 6

Carbonic Anhydrase Esterase Activity—Esterase activity of carbonic anhydrase was assessed using para-nitrophenylacetate (pNPA) as the substrate as described previously. Pocker, Y. & Stone, J. T. (1967) Biochemistry 6, 668-678. Briefly, 10 μl samples of carbonic anhydrase solution were added to 500 μl of Tris-HCl, pH 7.5 and 50 μl of 50 mM pNPA in acetonitrile. Kinetics of hydrolysis of pNPA was observed by the increase in absorbance at 400 nm due to the appearance of the paranitrophenolate ion (pNP⁻). In all cases, the observed hydrolysis rate in absorbance units per second (AU/s) under these conditions was constant (pseudo-zero order). Hydrolysis rates were corrected for the hydrolysis of pNPA by the buffer for each type of buffer used. Hydrolysis rates were converted to concentration of active protein via a standard curve constructed from dilutions of known concentrations of native protein. The active protein concentration data was reproducible to within 5-8% in replicated experiments.

### EXAMPLE 7

Modeling of Association and Dissociation—Transfer free energies for pairs of proteins into 1M arginine HCl and 1M guanidinium HCl solutions were computed by a method described previously. Baynes, B. M. & Trout, B. L. (2004) Biophys. J. 87, 1631-1639. Associating proteins were modeled as spheres 20 Å or as planes of surface area 400 πÅ². (While these shapes may seem like drastic approximations, interaction parameters used below to calculate additive effects were obtained from all-atom molecular simulation data.) The distance between the surfaces of the proteins in any configuration was defined as the reaction coordinate, x, for association and dissociation. The associated state was taken to be the point at which the proteins are in contact with each other (x=0), the dissociated state at infinite separation, and the transition state at a separation distance of 6 Å, or about one shell of water around each protein.

The free energy and the activation free energy of association were defined to be −8 and 2 kcal/mol, respectively. An empirical reaction coordinate-free energy surface between these points was constructed from Gaussian functions for the dimer and transition states and an inverse sixth power repulsive term (x<0). The exact function used was:

$$\begin{matrix} μ = - 9.05 {e}^{-} \text{?} + 1.98 {e}^{-} \text{?} + {( \frac{15}{x + 15} )}^{6} & ( 42 ) \\ \text{?} \text{indicates text missing or illegible when filed} & \end{matrix}$$

where μ is the free energy.

Additive-induced perturbations to this free energy function were computed via:

Δμ_{P}^{tr}=−RTc_{X}∫(e^{-<U}^{_{XP}}^{>/RT}−e^{-<U}^{_{WP}}^{>/RT})dV (43)

where Δμₚ^{tr} is the transfer free energy, RT is the gas constant times absolute temperature, cₓ is the additive concentration, U_{XP} is the additive-protein potential of mean force, U_{WP} is the water-protein potential of mean force, and the integral is over the solvent volume. The potentials of mean force were modeled as exponential-6 potentials and fit to radial distribution data obtained from all-atom molecular dynamics simulation. Baynes, B. M. & Trout, B. L. (2003) J. Phys. Chem. B 107, 14058-14067. The model for water was taken directly from. Baynes, B. M. & Trout, B. L. (2004) Rational design of solution additives for the preventing of protein aggregation, Biophys. J. 87, 1631-1639. Guanidinium was modeled as urea from the same reference, but with double the free energy change, since protein free energy effects due to guanidinium chloride are on average double that of urea. Myers, J. K., Pace, C. N., & Scholtz, J. M. (1995) Protein Sci. 4, 2138-2148. Arginine was modeled as having a characteristic radius of 4 Å and no effect on the free energy of the dissociated state.

### INCORPORATION OF REFERENCE

All of the U.S. patents and U.S. patent application publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A compound, comprising a non-protein-binding moiety (NPBM) and at least one protein-binding group (PBG).

2. The compound of claim 1, wherein the NPBM is a polyol, sugar, amino acid, or dendrimer moiety.

3. The compound of claim 1, wherein the NPBM is a polyol moiety; and said polyol moiety is a sorbitol or maimitol moiety.

4. The compound of claim 1, wherein the NPBM is a sugar moiety; and said sugar moiety is a glucose, sucrose, or trehalose moiety.

5. The compound of claim 1, wherein the NPBM is an amino acid moiety; and said amino acid moiety is an arginine betaine, proline, or ectoine moiety.

6. The compound of claim 1, wherein the NPBM is a dendrimer moiety; and said dendrimer moiety is based on benzene, pentaerythritol, P(CH₂OH)₃, or TRIS.

7. The compound of any of claims 1-6, wherein the PBG is a urea, guanidinium ion, detergent, amino acid, denaturant, surfactant, polysorbate, polaxamer, citrate, chaotrope, or acetate group.

8. The compound of any of claims 1-6, wherein the PBG is a guanidinium ion.

9. The compound of any of claims 1-6, wherein the PBG is sodium dodecyl sulfate.

10. A compound represented by formula I: 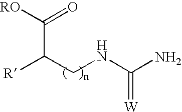
I
wherein:
R is an electron pair, H, alkyl, aryl, heteroaryl, aralkyl, heteroaralkyl, or an alkali metal;
R′ is H, alkyl, aryl, heteroaryl, aralkyl, heteroaralkyl, or (R″)₃N;
R″ is an electron pair, H, alkyl, aryl, heteroaryl, aralkyl, or heteroaralkyl;
W is O, NH₂⁺, (halogen)⁻, or S; and
n is 1, 2, or 4-100.

11. The compound of claim 10, wherein R is an electron pair.

12. The compound of claim 10, wherein R′ is H.

13. The compound of claim 10, wherein R′ is (R″)₃N.

14. The compound of claim 10, wherein R′ is H₃N⁺.

15. The compound of claim 10, wherein W is NH₂+Cl⁻.

16. The compound of claim 10, wherein n is 1.

17. The compound of claim 10, wherein n is 2.

18. The compound of claim 10, wherein n is 4.

19. The compound of claim 10, wherein n is 5.

20. The compound of claim 10, wherein n is 6.

21. The compound of claim 10, wherein R is an electron pair, R′ is H₃N⁺, W is NH₂⁺Cl⁻, and n is 1.

22. The compound of claim 10, wherein R is an electron pair, R′ is H₃N⁺, W is NH₂⁺Cl⁻, and n is 2.

23. The compound of claim 10, wherein R is an electron pair, R′ is H₃N⁺, W is NH₂⁺Cl⁻, and n is 4.

24. The compound of claim 10, wherein R is an electron pair, R′ is H₃N⁺, W is NH₂⁺Cl⁻, and n is 5.

25. The compound of claim 10, wherein R is an electron pair, R′ is H₃N⁺, W is NH₂⁺Cl⁻, and n is 6.

26. The compound of claim 10, wherein R is an electron pair, R′ is H₃N⁺, W is O, and n is 1.

27. The compound of claim 10, wherein R is an electron pair, R′ is H₃N⁺, W is O, and n is 2.

28. The compound of claim 10, wherein R is an electron pair, R′ is H₃N⁺, W is O, and n is 4.

29. The compound of claim 10, wherein R is an electron pair, R′ is H₃N⁺, W is O, and n is 5.

30. The compound of claim 10, wherein R is an electron pair, R′ is H₃N⁺, W is O, and n is 6.

31. The compound of claim 10, wherein R is an electron pair, R′ is H, W is NH₂⁺Cl⁻, and n is 1.

32. The compound of claim 10, wherein R is an electron pair, R′ is H, W is NH₂⁺Cl⁻, and n is 2.

33. The compound of claim 10, wherein R is an electron pair, R′ is H+, W is NH₂⁺Cl⁻, and n is 4.

34. The compound of claim 10, wherein R is an electron pair, R′ is H, W is NH₂⁺Cl⁻, and n is 5.

35. The compound of claim 10, wherein R is an electron pair, R′ is H, W is NH₂⁺Cl⁻, and n is 6.

36. The compound of claim 10, wherein R is an electron pair, R′ is H, W is O, and n is 1.

37. The compound of claim 10, wherein R is an electron pair, R′ is H, W is O, and n is 2.

38. The compound of claim 10, wherein R is an electron pair, R′ is H, W is O, and n is 4.

39. The compound of claim 10, wherein R is an electron pair, R′ is H, W is O, and n is 5.

40. The compound of claim 10, wherein R is an electron pair, R′ is H, W is O, and n is 6.

41. A compound selected from the group consisting of: 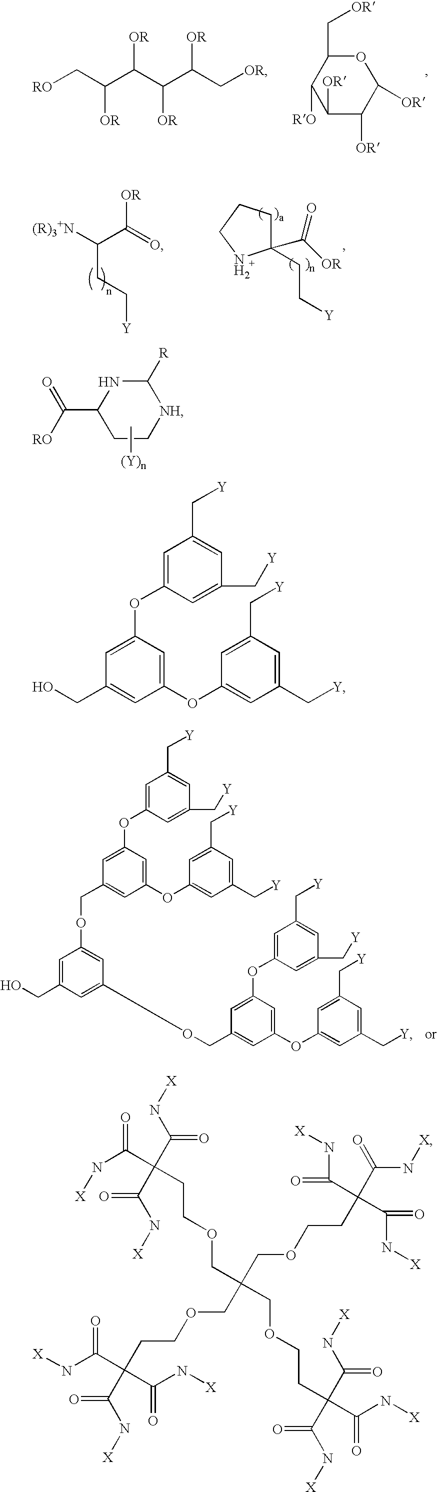
wherein, independently for each occurrence,
R is an electron pair, H, alkyl, aryl, heteroaryl, aralkyl, heteroaralkyl, an alkali metal, or CH₂Y;
R′ is H, a sugar radical, or CH₂Y;
n is an integer from 1 to 100, inclusive;
a is 1, 2, or 3;
X is C(CH₂Y)₃; and
Y is a protein binding group,
wherein at least one Y is present in all compounds.

42. The compound of claim 41, wherein Y is a guanidinium ion.

43. A polymer of formula II, III, IV, V, VI, VII, VIII, or IX: 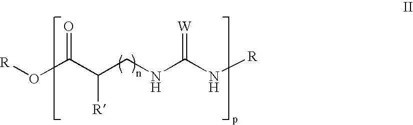
wherein, independently for each occurrence:
R is an electron pair, H, alkyl, aryl, heteroaryl, aralkyl, heteroaralkyl, or an alkali metal;
R′ is H, alkyl, aryl, heteroaryl, aralkyl, heteroaraklyl, or (R″)₃N;
R″ is an electron pair, H, alkyl, aryl, heteroaryl, aralkyl, or heteroaralkyl,
W is O, NH₂⁺(halogen)⁻, or S;
n is 1, 2, or 4 -100; and
p is an integer from 2 to 1000 inclusive; 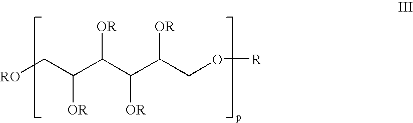
wherein, independently for each occurence;
R is H, alkyl, aryl, heteroaryl, aralkyl, heteroaralkyl, or an alkali metal, or CH₂Y;
p is an integer from 2 to 1000 inclusive; and
Y is a PBG, wherein at least one Y is present; 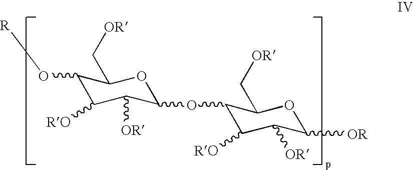
wherein, independently for each occurrence:
R is H, alkyl, aryl, heteroaryl, aralkyl, heteroaralkyl, or an alkali metal, or CH₂Y;
R′ is H, alkyl, aryl, heteroaryl, aralkyl, heteroaralkyl, or (R″)₃N;
R″ is an electron pair, H, alkyl, aryl, heteroaryl, aralkyl, or heteroaralkyl;
p is an integer from 2 to 1000 inclusive; and
Y is a PBG, wherein at least one Y is present; 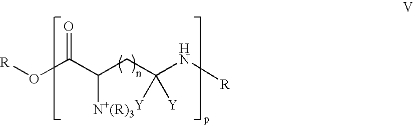
wherein, independently for each occurrence:
R is H, alkyl, aryl, heteroaryl, aralkyl, heteroaralkyl, or an alkali metal, or CH₂Y;
n is an integer from 1 to 100 inclusive;
p is an integer from 2 to 1000 inclusive; and
Y is a PBG; 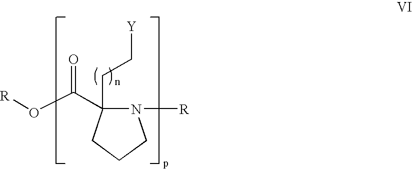
wherein, independently for each occurrence,
R is H, alkyl, aryl, heteroaryl, aralkyl, heteroaralkyl, an alkali metal, or CH₂Y;
n is an integer from 1 to 100, inclusive;
a is 1,2, or 3;
Y is a PBG; and
p is an integer from 2 to 1000, inclusive; 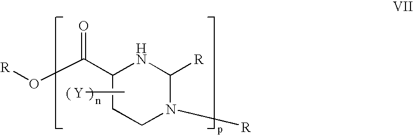
wherein, independently for each occurrence,
R is H, alkyl, aryl, heteroaryl, aralkyl, heteroaralkyl, an alkali metal, or CH₂Y;
n is an integer from 1 to 6, inclusive;
Y is a PBG; and
p is an integer from 2 to 1000, inclusive; or 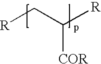
VIII
wherein, independently for each occurrence,
R is H, OH, alkyl, alkoxy, aryl, heteroaryl, aralkyl, heteroaralkyl, —O-alkali metal, CH₂Y, OCH₂Y, or has a structure selected from the following: 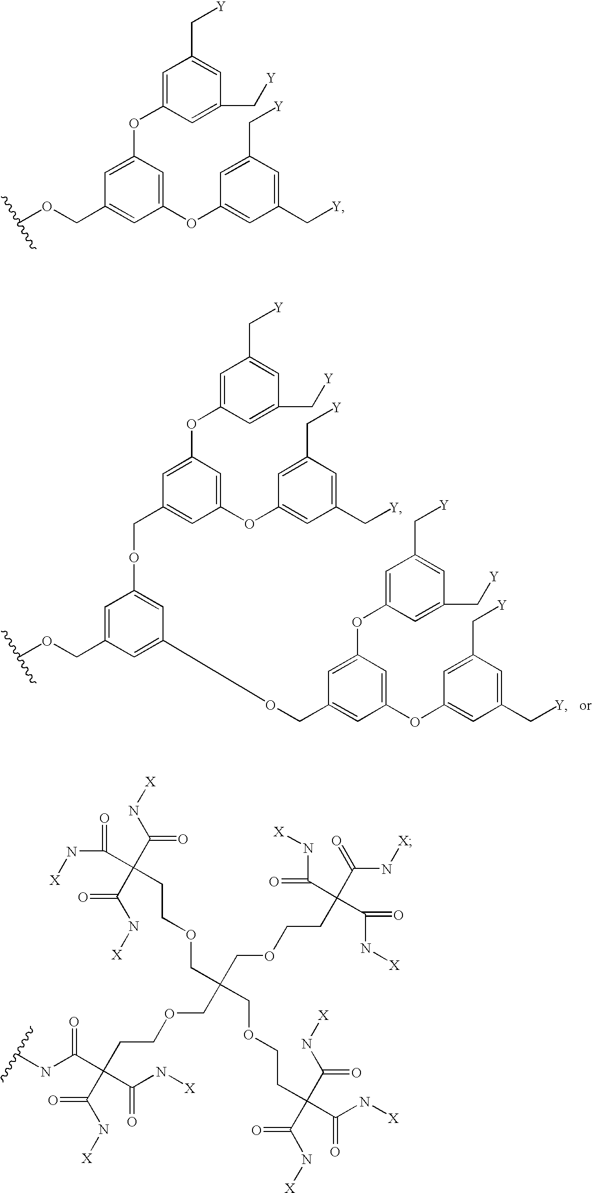
a is 1,2, or 3;
X is C(CH₂Y)₃;
Y is a PBG, wherein at least one Y is present; and
p is an integer from 2 to 1000, inclusive; or 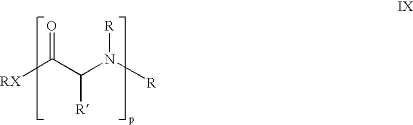
wherein, individually for each occurrence:
R is an electron pair, H, alkyl, aryl, heteroaryl, aralkyl, heteroaralkyl, or an alkali metal;
R′ is a side chain of an alpha-amino acid, wherein at least one instance of R′ is the side chain of arginine;
X is O or NR; and
p is an integer from 2 to 1000, inclusive.

44. A method of screening compounds or polymers for the property of inhibiting protein aggregation in solution, comprising:
a) computing a set of parameters utilizing molecular modeling based on compounds or polymers known to have the property of inhibiting protein aggregation;
b) applying those parameters to other compounds or polymers; and
c) choosing the compounds or polymers that meet the criteria of those parameters.

45. A method of preparing a compound or polymers having the property of protein aggregation inhibition in solution, comprising:
a) computing a set of parameters utilizing molecular modeling based on compounds or polymers known to have the property of inhibiting protein aggregation;
b) designing a compound or polymer having the property of protein aggregation inhibition in solution based on those parameters; and
c) synthesizing the compound or polymer having the property of protein aggregation inhibition in solution.

46. A method of classifying a compound or polymer as either inhibitory of protein aggregation in solution or not inhibitory of protein aggregation in solution, comprising:
a) computing a set of parameters utilizing molecular modeling based on compounds or polymers known to have the property of inhibiting protein aggregation;
b) applying those parameters to a compound or polymer; and
c) classifying the compound or polymer that meet the criteria of those parameters as inhibitory of protein aggregation in solution.

47. A method of determining the preferential binding coefficient, Γ_{XP}, of an additive in a protein solution, comprising:
a) determining the phase space trajectories of the protein, solvent, and additive using molecular dynamics;
b) calculating the distance, r, between the center of mass for both the solvent molecule and additive molecule to the protein's van der Waals surface;
c) determining the minimum distance, r*, at which no significant differences between the local (r=r*) and bulk density are observed;
d) determining which molecules lie within the distance, r*, from the protein surface and classifying these molecules as the local domain;
e) determining which molecules lie outside the distance, r*, from the protein surface and classifying these molecules as the bulk domain;
f) determining the instantaneous preferential binding coefficient, Γ_{XP} (t), using the following formula:
Γ_{XP}(t)=n^{II}_{X}−n^{I}_{X}(n^{II}_{W}/n^{I}_{w})
wherein:
n^{II}ₓ=the number of additive molecules in the bulk domain;
n^{I}ₓ=the number of additive molecules in the local domain;
n^{II}ₓ=the number of solvent molecules in the bulk domain; and
n^{I}_{w}=the number of solvent molecules in the local domain; and
g) calculating the preferential binding coefficient, Γ_{XP}, as the time average of each of the values in step f) using the following formula:
$${Γ}_{XP} = \frac{1}{t} {\int }_{0}^{t} {Γ}_{XP} ( {t}^{′} ) d {t}^{′} .$$

48. A method of suppressing or preventing aggregation of a protein in solution, comprising the step of combining in a solution the compound or polymer of any of claims 1 to 43 and a protein.

49. The method of claim 48, wherein the protein is a recombinant protein.

50. The method of claim 48, wherein the protein is a recombinant antibody.

51. The method of claim 48, wherein the protein is a recombinant human antibody.

52. The method of claim 48, wherein the protein is a recombinant human protein.

53. The method of claim 48, wherein the protein is recombinant human insulin, recombinant human erythropoietin or a recombinant human interferon.

54. The method of claim 48, wherein the solution is an aqueous solution.

55. The method of claim 48, wherein the protein is a recombinant protein; and the solution is an aqueous solution.

56. The method of claim 48, wherein the protein is a recombinant human protein; and the solution is an aqueous solution.

57. A method of decreasing the toxicological risk associated with administering a protein to a mammal in need thereof, comprising the steps of adding to a first solution of a protein a compound or polymer of any of claims 1 to 43 to give a second solution; and administering to a mammal in need thereof a therapeutic amount of said second solution.

58. The method of claim 57, wherein the protein is a recombinant protein.

59. The method of claim 57, wherein the protein is a recombinant antibody.

60. The method of claim 57, wherein the protein is a recombinant human antibody.

61. The method of claim 57, wherein the protein is a recombinant mammalian protein.

62. The method of claim 57, wherein the protein is a recombinant human protein.

63. The method of claim 57, wherein the protein is recombinant human insulin, recombinant human erythropoietin or a recombinant human interferon.

64. The method of claim 57, wherein the first solution and the second solution are aqueous solutions.

65. The method of claim 57, wherein the protein is a recombinant protein; and the first solution and the second solution are aqueous solutions.

66. The method of claim 57, wherein the protein is a recombinant human antibody; and the first solution and the second solution are aqueous solutions.

67. The method of claim 57, wherein the protein is a recombinant human protein; and the first solution and the second solution are aqueous solutions.

68. A method of facilitating native folding of a recombinant protein in solution, comprising the step of combining in a solution a compound or polymer of any of claims 1 to 43 and a recombinant protein.

69. The method of claim 68, wherein the recombinant protein is a recombinant antibody.

70. The method of claim 68, wherein the recombinant protein is a recombinant human antibody.

71. The method of claim 68, wherein the recombinant protein is a recombinant mammalian protein.

72. The method of claim 68, wherein the recombinant protein is a recombinant human protein.

73. The method of claim 68, wherein the recombinant protein is recombinant human insulin, recombinant human erythropoietin or a recombinant human interferon.

74. The method of claim 68, wherein the solution is an aqueous solution.

75. The method of claim 68, wherein the recombinant protein is a recombinant human antibody; and the solution is an aqueous solution.

76. The method of claim 68, wherein the recombinant protein is a recombinant human protein; and the solution is an aqueous solution.
